(19) United States
(12) Patent Application Publication
(10) Pub. No.: US 2017/0325878 A1
(43) Pub. Date: Nov. 16, 2017

(54) SUCTION AND IRRIGATION SEALING GRASPER

(71) Applicant: Ethicon LLC, Guaynabo, PR (US)

(72) Inventors: Jeffrey D. Messerly, Cincinnati, OH (US); Peter K. Shires, Hamilton, OH (US); Chad P. Boudreaux, Cincinnati, OH (US); Edward G. Chekan, Chapel Hill, NC (US); Foster B. Stulen, Mason, OH (US); Shan Wan, Mason, OH (US); Cory G. Kimball, Hamilton, OH (US)

(21) Appl. No.: 15/493,427

(22) Filed: Apr. 21, 2017

Related U.S. Application Data
(60) Provisional application No. 62/334,790, filed on May 11, 2016.

(51) Int. Cl. A61B 18/14; A61M 1/00; A61M 3/02; A61B 18/12; A61B 18/00; A61B 17/00

(52) U.S. Cl. CPC A61B 18/1445; A61B 18/1206; A61M 1/0035; A61M 3/0258; A61M 1/0064; A61B 2018/00589; A61B 2017/00154; A61B 2018/0063; A61B 2018/126; A61B 2218/002; A61B 2218/007; A61B 2018/1253

(57) ABSTRACT

Aspects of the present disclosure are presented for a single surgical instrument configured for performing sealing procedures on tissues using electrosurgical or ultrasonic energy, irrigating tissue at the sealing site, and evacuating material from the sealing site. An end effector of the surgical instrument may include multiple members arranged in various configurations to collectively perform the aforementioned functions. The evacuation and irrigation elements may comprise one or more fluid paths configured to remove material from or deliver fluid to a surgical field. The instrument may include electrical and mechanical aspects that may be used to control the evacuation and irrigation functions. In this way, a user may use a single surgical instrument to perform these tasks during a tissue sealing procedure.

### STATEMENT OF PRIORITY

This application claims priority to and benefit of U.S. Provisional Application Ser. No. 62/334,790 entitled “Suction and Irrigation Sealing Grasper,” which was filed on May 11, 2016, the entirety of which is incorporated herein by reference and for all purposes.

### TECHNICAL FIELD

The present disclosure is related generally to medical devices with various mechanisms for grasping and sealing tissue. In particular, the present disclosure is related to medical devices with grasping instruments that perform sealing procedures and also include evacuation and irrigation functionality in the same device.

### BACKGROUND

In many surgeries, multiple devices are used to perform a combination of sealing a tissue (e.g., using electrosurgical energy or in other cases ultrasonic energy), evacuating material proximal to the sealing site, and irrigating the sealing site. A surgeon may hold at least one device for performing at least one of these functions, for example, in the offhand. Assistance is typically needed to enable the surgeon to perform these multiple functions without losing concentration on the surgical site. It is therefore desirable to provide a single surgical instrument configured to perform these multiple functions to aide the surgeon and increase performance, accuracy and safety during the surgery.

While several devices have been made and used, it is believed that no single prior device incorporates all of the functions disclosed herein, including a tissue sealing function, a fluid irrigation function, and a material evacuation function as recited in the appended claims.

### BRIEF SUMMARY

In some aspects, a surgical instrument is provided.

In one aspect, a surgical instrument may include a handle assembly, a shaft coupled to a distal end of the handle assembly, an end effector coupled to a distal end of the shaft, and a clamp arm coupled to the first jaw and configured to open the first jaw about a hinge coupled to the shaft. The end effector may include a first jaw, a second jaw, an evacuation mechanism configured to evacuate fluid, and an irrigation mechanism configured to transmit fluid. The first jaw and the second jaw may cooperate to capture tissue therebetween and at least one of the first and second jaws is configured to transmit electrosurgical energy to coagulate the tissue;

In one aspect, the surgical instrument further includes a spring torsion system configured to close the first jaw onto the second jaw while no force is exerted on the clamp arm.

In one aspect, the surgical instrument further includes a rotatable mechanical bar pivotally coupled to the clamp arm on a first end and pivotally coupled to a horizontal bar of the spring torsion system on the second end opposite the first end.

In one aspect of the surgical instrument, the rotatable mechanical bar is positioned at a first angle greater than 45 degrees and less than 90 degrees from horizontal when the first jaw is closed onto the second jaw.

In one aspect of the surgical instrument, the rotatable mechanical bar is positioned at a second angle less than 45 degrees and greater than 0 degrees from horizontal when the clamped arm is pressed and the first jaw is in an open position.

In one aspect, a control system of a surgical instrument may include a fluid source, a power generator, a pump coupled to the power generator and fluidically coupled to the fluid source, a valve, a vacuum source coupled to the valve, and a controller coupled to the valve. The surgical instrument my be fluidically coupled to the fluid source via a first fluid line, fluidically coupled to the pump via a second fluid line, fluidically coupled to the vacuum source via a first vacuum line, and fluidically coupled to the valve via a second vacuum line. The surgical instrument may also be electrically coupled to the generator via a first electrical line, and electrically coupled to the controller via a second electrical line.

In one aspect of the control system, the surgical instrument includes a first button configured to control the generator to cause the pump to deliver a modulated drip functionality of fluid to the surgical instrument from the fluid source via the second fluid line, and simultaneously cause the controller to control the valve to deliver a pulsing vacuum function to the surgical instrument from the vacuum source via the second vacuum line.

In one aspect of the control system, the surgical instrument may further include a second button configured to cause an uninterrupted vacuum functionality directly to the surgical instrument from the vacuum source via the first vacuum line.

In one aspect of the control system, the surgical instrument may further include a third button configured to cause an uninterrupted fluid flow functionality directly to the surgical instrument from the fluid source via the first fluid line.

In one aspect of the control system, a rate of drip of the fluid in the modulated drip functionality may be controlled by the generator and is directly proportional to a rate of radio frequency (RF) energy delivered by the generator to the surgical instrument.

In one aspect of the control system, a rate of vacuum pulsing of the pulsing vacuum function is controlled by the controller and is directly proportional to a rate of radio frequency (RF) energy delivered by the generator to the surgical instrument.

In one aspect, a surgical device may include a handle including a power control member, an irrigation control member, and an evacuation control member, an end effector including two electrodes of opposite polarities, an evacuation lumen, and an irrigation lumen, and an elongate member connecting the handle and the end effector.

In one aspect, the surgical device may have an effective distance between a distal end of one or both of the two electrodes and a distal end of the evacuation lumen.

In one aspect of the surgical device, the distal end of the evacuation lumen is located proximally to the distal end of the one or both of the two electrodes, and the effective distance has a range of 0.1 inch to 0.5 inches.

In one aspect of the surgical device, the distal end of the evacuation lumen is located proximally to the distal end of the one or both of the two electrodes, and wherein the effective distance has a range of 0.2 inches to 0.4 inches.

In one aspect of the surgical device, the distal end of the evacuation lumen is located proximally to the distal end of the one or both of the two electrodes, and wherein the effective distance has a range of 0.3 inches.

In one aspect, a surgical device may include a handle having a power control member, an irrigation control member, and an evacuation control member, an end effector including at least two electrodes of opposite polarities, an evacuation lumen, and an irrigation lumen, a boot around the end effector, and an elongate member connecting the handle and the end effector.

In one aspect of the surgical device, the boot includes an opening on a side surface of the boot.

In one aspect of the surgical device, the boot includes one or more ribs on a side surface.

In one aspect of the surgical device, the boot includes an S-wave portion on a side surface.

In one aspect of the surgical device, the boot has a shape that is configured to change to conform to a tissue surface when pressed thereagainst.

In an aspect, an end effector of a surgical device includes a first jaw, a second jaw, a closure saddle in mechanical communication with the first jaw, a closure tube in mechanical communication with the closure saddle, an evacuation mechanism configured to evacuate fluid, and an irrigation mechanism configured to transmit fluid. The first jaw and the second jaw may cooperate to capture a tissue therebetween and at least one of the first jaw and the second jaw is configured to transmit electrosurgical energy to coagulate the tissue. The closure saddle my be configured to adjust a position of the first jaw with respect to the second jaw based on a position of the closure tube.

In one aspect of the end effector, the position of the first jaw with respect to the second jaw is a closed position when the closure tube is in a proximal position.

In one aspect of the end effector, the position of the first jaw with respect to the second jaw is an open position when the closure tube is in a distal position.

In one aspect of the end effector, the closure tube is configured to translate in a longitudinal direction.

In one aspect, the end effector further includes a pivot pin in mechanical communication with the first jaw in which the first jaw is configured to pivot about an axis of the pivot pin.

In one aspect, the end effector further includes a cam configured to engage a portion of the closure saddle.

In one aspect of the end effector, at least one of the first jaw and the second jaw comprises one or more insulated pins configured to separate and insulate the first jaw and the second jaw when the first jaw and the second jaw are in a closed position.

The foregoing summary is illustrative only and is not intended to be in any way limiting. In addition to the illustrative aspects, aspects, and features described above, further aspects, aspects, and features will become apparent by reference to the drawings and the following detailed description.

### BRIEF DESCRIPTION OF THE DRAWINGS

The features of the aspects described herein are set forth with particularity in the appended claims. The aspects, however, both as to organization and methods of operation may be better understood by reference to the following description, taken in conjunction with the accompanying drawings as follows.

FIG. 1A depicts aspects of a medical device, having a fluid control system according to various aspects.

FIG. 1B depicts a more detailed view of the end effector of the medical device depicted in FIG. 1A.

FIG. 1C depicts a schematic of one aspect of a fluid control system.

FIG. 1D depicts a cross-sectional view of the evacuation and irrigation mechanisms of the medical device depicted in FIG. 1A.

FIG. 1E depicts a perspective view of the cross-sectional view of the evacuation and irrigation mechanisms of the medical device depicted in FIG. 1D.

FIG. 1F depicts a cross-sectional view of the end effector of the medical device depicted in FIG. 1A.

FIG. 1G depicts an example of evacuation and irrigation tubes, as well as a power cable connected, to the surgical device depicted in FIG. 1A.

FIG. 2 depicts one example design of an end effector including jaw members for grasping and applying sealing energy and according to some aspects, including first and second jaw members and an evacuation and irrigation path included in one of the first and second jaw members.

FIG. 3 depicts the end effector of FIG. 2 in an open position.

FIG. 4 depicts the end effector of FIG. 2 with a semi transparent view, thereby depicting some further details of performing opening and closing of the jaws, according to some aspects.

FIG. 5 depicts a longitudinal cross-sectional view of the end effector in FIG. 2.

FIGS. 6-8 depict the end effector of FIG. 2 in various exploded views.

FIG. 9 depicts another example of a medical instrument with bipolar jaws on the end of an evacuation wand.

FIGS. 10A and 10B depict a use of the medical instrument depicted in FIG. 9.

FIG. 11 depicts a first cross-sectional interior view of the medical instrument depicted in FIG. 9 having closed jaws of an end effector.

FIG. 12 depicts a second cross-sectional interior view of the medical instrument depicted in FIG. 9 having open jaws of an end effector.

FIGS. 13 and 14 depict expanded cross-sectional interior views of the medical instrument as depicted in FIGS. 11 and 12, respectively.

FIG. 15 depicts an expanded perspective view of the distal end of the medical device depicted in FIG. 11.

FIG. 16 depicts an expanded perspective view of the distal end of the medical device depicted in FIG. 12.

FIG. 17 is a block system diagram of a control system for fluid irrigation and aspiration through a medical device, according to some aspects.

FIG. 18 is a block diagram of electrical components suitable for a use with a surgical system as depicted in FIG. 1A.

FIGS. 19A-19E depict a second aspect of a surgical device.

FIG. 19F is a flow chart illustrating functions of control components of a surgical device according to some aspects.

FIGS. 20A-20D, 21A, 21B, 22A-22D, 23A-23D, 24A-24C, 25A, and 25B illustrate various aspects and uses of a surgical device having a flexible boot around an end effector.

### DETAILED DESCRIPTION

In the following detailed description, reference is made to the accompanying drawings, which form a part hereof. In the drawings, similar symbols and reference characters typically identify similar components throughout the several views, unless context dictates otherwise. The illustrative aspects described in the detailed description, drawings, and claims are not meant to be limiting. Other aspects may be utilized, and other changes may be made, without departing from the scope of the subject matter presented here.

The following description of certain examples of the technology should not be used to limit its scope. Other examples, features, aspects, aspects, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, aspects, examples, etc., described herein may be combined with any one or more of the other teachings, expressions, aspects, examples, etc., that are described herein. The following-described teachings, expressions, aspects, examples, etc., should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Also, in the following description, it is to be understood that terms such as front, back, inside, outside, top, bottom, and the like are words of convenience and are not to be construed as limiting terms. Terminology used herein is not meant to be limiting insofar as devices described herein, or portions thereof, may be attached or utilized in other orientations. The various aspects will be described in more detail with reference to the drawings. Throughout this disclosure, the term “proximal” is used to describe the side of a component, e.g., a shaft, a handle assembly, etc., closer to a user operating the surgical instrument, e.g., a surgeon, and the term “distal” is used to describe the side of the component further from the user operating the surgical instrument.

Aspects of the present disclosure are presented for a single surgical instrument configured for grasping tissue, performing sealing procedures using electrosurgical or ultrasonic energy, evacuating, and providing irrigation. An end effector of the surgical instrument may include multiple members arranged in various configurations to collectively perform the aforementioned functions. The evacuation and irrigation elements may comprise one or more fluid paths configured to deliver fluid to or evacuate fluid from a surgical field. In certain aspects, the fluid may comprise any fluid, including a gas, liquid, combination of the two, as well as fluids that may further include particulates, e.g., electrosurgical smoke. In this way, a user, such as a clinician or surgeon, may rely on a single surgical instrument to perform these tasks typical in surgery while having an extra hand available and so as to not need to divert his or her concentration away from the surgical site in order to access multiple devices. Further, the timing for performing each of the functions may be made quicker due to not needing to switch using multiple devices.

In some aspects, an end effector of a surgical instrument includes a pair of jaws for grasping and applying electrosurgical (e.g., radio frequency (“RF”)) energy to tissue at a surgical site. A first jaw may also include an evacuation and irrigation path. An insulating layer or over mold may be included in between the two jaws to allow for one jaw to supply energy at a first pole and the other jaw to supply energy at a second pole. In some aspects, the jaw including the evacuation and irrigation path may also include small holes on the sides or top to allow for evacuation through the sides or top. These features may allow for spot coagulation and evacuating.

In some aspects, an end effector of a surgical instrument includes a pair of jaws for grasping and applying electrosurgical energy to tissue at the surgical site. The pair of jaws may also form an evacuation and irrigation path when the jaws are closed. An insulated member may be included in between the pair of jaws and may also include an irrigation and evacuation path within. In some aspects, the insulated member may be shaped like a wedge such that translation of the insulated member in a longitudinal direction parallel to the shaft coupled to the end effector may cause the jaws to open and close.

In some aspects, an end effector of a surgical instrument includes an ultrasonic member and an irrigation and evacuation tube. The ultrasonic member may be implemented in various different shapes, such as in a spoon shape, a hook shape, a wedge shape, or in a shape configured to grab or grasp tissue. The ultrasonic member may be configured to deliver ultrasonic energy through being vibrated at an ultrasonic frequency. The irrigation and evacuation tube may be located near to the ultrasonic member at the end effector. In some aspects, one or both of the ultrasonic member and the irrigation and evacuation tube may be retracted into a closure tube to allow for focused use of one or the other members. In other cases, the irrigation and evacuation tube may be built into the ultrasonic member, such as by having a hole carved out of part of the ultrasonic member and a tube connected therefrom.

In some aspects, any of the aforementioned examples may also be configured to articulate along at least one axis through various means, including, for example, a series of joints, one or more hinges, and one or more cam or pulley systems. Other various features may include cameras or lights coupled to one or more of the members of the end effector, and monopolar or bipolar options for the electrosurgical devices.

Various features described herein may be incorporated in electrosurgical devices for applying electrical energy to tissue in order to treat and/or destroy the tissue are also finding increasingly widespread applications in surgical procedures. An electrosurgical device typically includes a hand piece, an instrument having a distally-mounted end effector (e.g., one or more electrodes). The end effector can be positioned against the tissue such that electrical current is introduced into the tissue. Electrosurgical devices can be configured for bipolar or monopolar operation. During bipolar operation, current is introduced into and returned from the tissue by active and return electrodes, respectively, of the end effector. During monopolar operation, current is introduced into the tissue by an active electrode of the end effector and returned through a return electrode (e.g., a grounding pad) separately located on a patient's body. Heat generated by the current flowing through the tissue may form hemostatic seals within the tissue and/or between tissues and thus may be particularly useful for sealing blood vessels, for example. The end effector of an electrosurgical device may also include a cutting member that is movable relative to the tissue and the electrodes to transect the tissue.

Electrical energy applied by an electrosurgical device can be transmitted to the instrument by a generator in communication with the hand piece. The electrical energy may be in the form of radio frequency (“RF”) energy. RF energy is a form of electrical energy that may be in the frequency range of 200 kilohertz (kHz) to 1 megahertz (MHz). In application, an electrosurgical device can transmit low frequency RF energy through tissue, which causes ionic agitation, or friction, in effect resistive heating, thereby increasing the temperature of the tissue. Because a sharp boundary is created between the affected tissue and the surrounding tissue, surgeons can operate with a high level of precision and control, without sacrificing un-targeted adjacent tissue. The low operating temperatures of RF energy is useful for removing, shrinking, or sculpting soft tissue while simultaneously sealing blood vessels. RF energy works particularly well on connective tissue, which is primarily comprised of collagen and shrinks when contacted by heat.

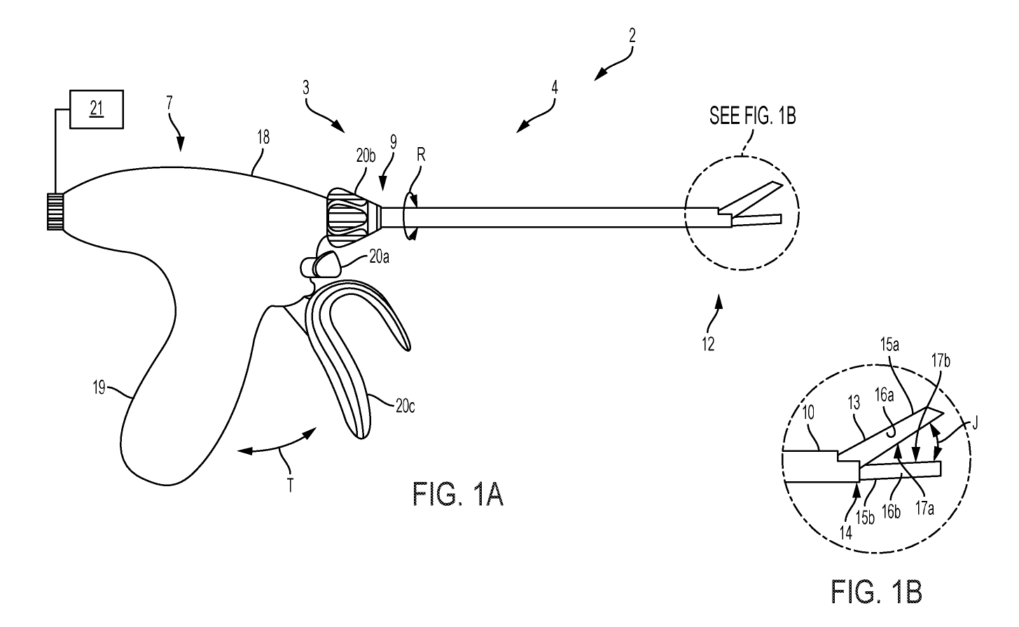

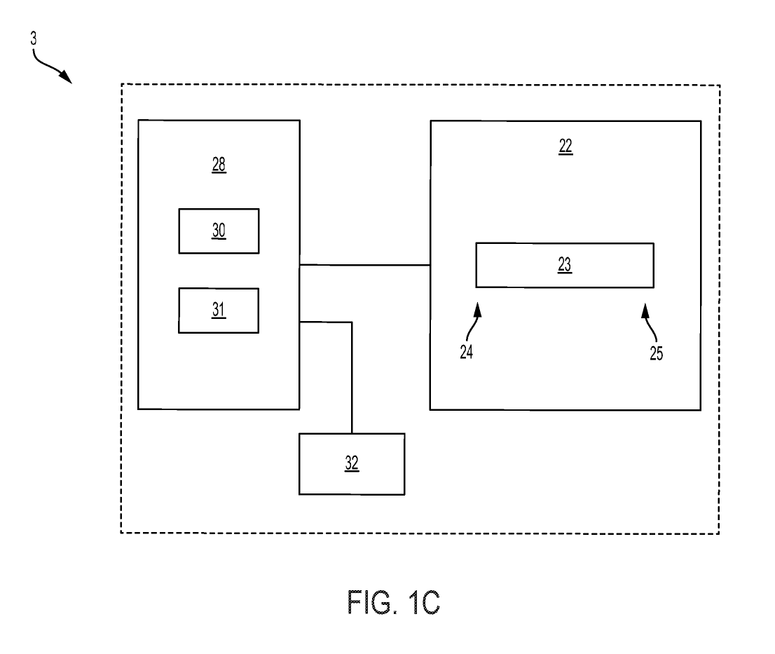

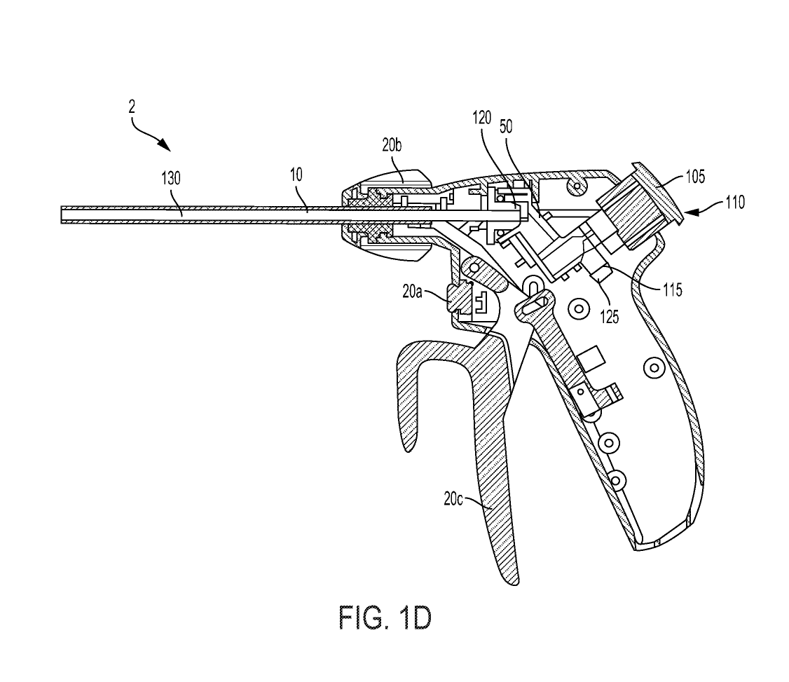

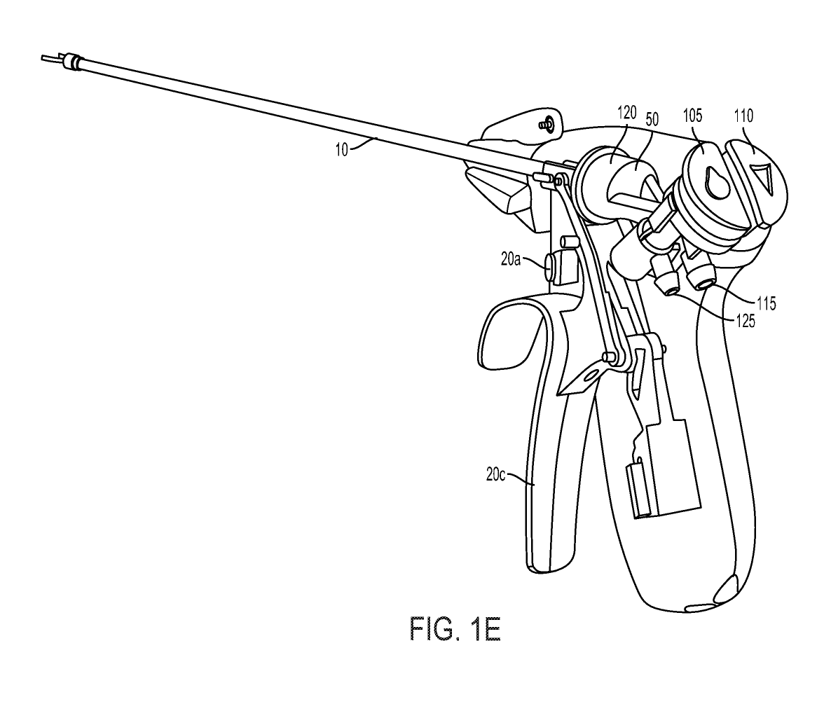

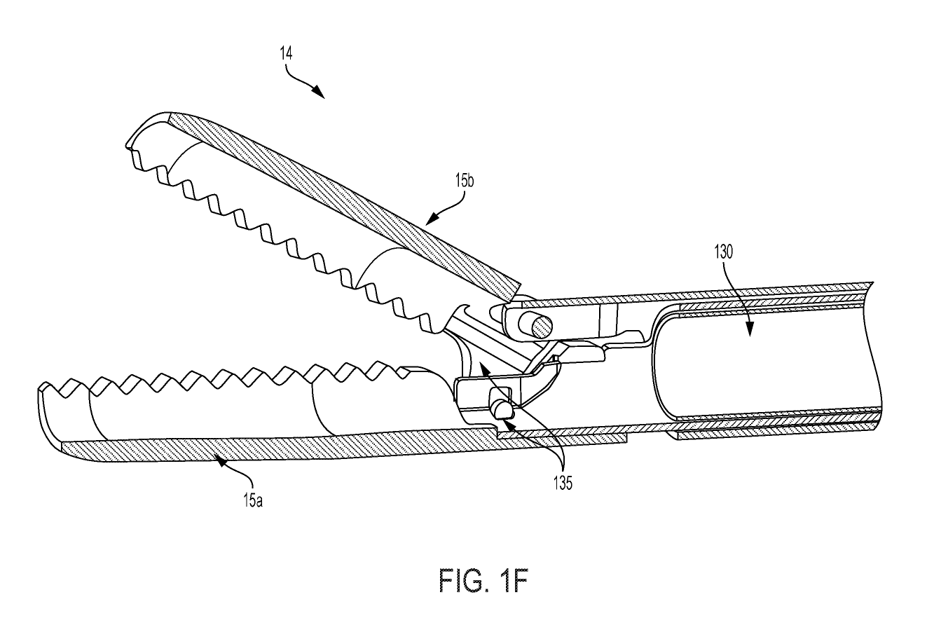

Referring to FIG. 1A, a medical device 2 is illustrated, configurable with a fluid control system 3 according to various aspects. The medical device 2 comprises an elongate member 4, such as a shaft, having a proximal portion 9 coupled to a handle assembly 7. A distal portion 12 of the elongate member 4 comprises an end effector 14 (see FIG. 1B) coupled to a distal end 14 of the shaft 10. In some aspects, the end effector 14 comprises a first jaw 15a and a second jaw 15b, each having an outer portion or surface 16a, 16b. At least one of the first jaw 15a and the second jaw 15b is rotatably movable relative to the other along a path depicted by arrow J to transition the jaws 15a, 15b between open and closed positions. In operation, the jaws 15a, 15b may be transitioned from the open position to a closed position to capture tissue therebetween. Captured tissue may contact one or more working portions of the jaw set, indicated generally as 17a, 17b, configured to apply energy, e.g., bipolar energy, to treat target tissue. In some aspects, the first jaw 15a or the second jaw 15b may include an irrigation and evacuation path.

The handle assembly 7 comprises a housing 18 defining a grip 19. In various aspects, the handle includes one or more control interfaces 20a-c, e.g., a button or switch 20a, rotation knob 20b rotatable along arrow R, and a trigger 20c movable relative to the grip 19 along arrow T, configured to provide operation instructions to the end effector 13. Multiple buttons, knobs, or triggers described may also be included as part of the housing 18 in order to manipulate one or more of the functioning members at the end effector 14. In some aspects, the handle assembly 7 is further configured to electrically couple to an energy source 21 to supply the medical device 2 with energy. While the energy source 21 is illustrated as generally coupled to the handle assembly 7, e.g., with a cord, it is to be understood that in some aspects the energy source 21 may be positioned within the elongate member 4. For example, in one aspect, the energy source 21 comprises one or more direct current batteries positioned in the handle 7, shaft 10, or a portion thereof.

As introduced above, the medical device 2 includes or is configurable with the fluid control system 3 to control fluid, e.g., smoke, steam, or other fluid. FIG. 1C depicts a schematic of one aspect of a fluid control system 3. The fluid control system 3 includes a fluid path element 22 comprising one or more fluid paths 23. The one or more fluid paths 23 may be fluidically coupled to one or more proximal fluid ports 24 and one or more distal fluid ports 25. With further reference to FIG. 1A, the one or more fluid paths 23 may extend along a portion of the shaft 10 and, in various aspects, may further extend along the handle 7, end effector 14, or only along a portion of the end effector 14 or shaft 10. In certain aspects, the fluid paths 23 may be defined by lumens, lines, channels, voids, ducts, cavities, or tubing which may be externally or internally positioned relative to the handle 7, shaft 10, or end effector 14 or may be integrally formed within such components of the medical device 2. For example, the fluid paths 23 may be integrated into the housing 18 of the handle 7, shaft 10, or end effector 14 or may comprise fluid paths configured as accessory features such as a cover, mold, attachment, sleeve, coating, or the like, that may be positioned on or associated with the handle 7, shaft 10, or end effector 14.

As introduced above, the fluid control system 3 may further comprise or be configured to fluidically couple to a fluid supply and transport element 28 comprising a supply component 30 and a transport component 31. The supply component 30 is configured to supply or receive fluid from the fluid path element 22 and may comprise a fluid source to supply fluid to a fluid path element 23 or a fluid reservoir, which may comprise an environment external to the fluid path element 23 to receive fluid from the fluid path element 22. The transport component 31 is configured to move fluid through the one or more fluid paths of the fluid path element 22. In various aspects, the transport component 31 is configured to move fluid passively through the fluid path element 23 via gravity or diffusion, for example, and thus may not comprise a physical structure. In various aspects, the transport component 31 comprises a pump or pressure differential configured to actively move or transport fluid through the fluid path element 22. For example, the transport component 31 may include a pressurized or compressed fluid supply or a pump to pressurize or compress the fluid supply. In one aspect, the fluid supply system 3 includes a valve positioned between the supply component 30 and the fluid path element 22. Fluid path through the valve may be controlled to control transport of fluid through the one or more fluid paths. For example, the transport component 31 may comprise or generate a pressure differential between two outlets of the valve such that fluid is motivated to flow through the valve when the valve is open.

As previously described, the one or more fluid paths 23 may be fluidically coupled to one or more proximal fluid ports 24 and one or more distal fluid ports 25. The proximal fluid ports 24 may be positioned along the elongate member 4, e.g., within or adjacent to the handle 7, shaft 10, or end effector 14. The distal fluid ports 25 may be configured and positioned to deliver or intake fluid from the surgical field or tissue treatment site adjacent the distal portion 12 of the elongate member 4, e.g., the distal end of the shaft 10, the end effector 14, or working portion thereof 17a, 17b.

In various aspects, the fluid control system 3 includes or is configured to associate with an activation element 32. The activation element 32 may be operatively coupled to the fluid supply and transport element 28 to activate the transport component 31 to, for example, provide power to a pump or to open a valve or port. In some aspects, the activation element 32 comprises a switch electrically coupled to the energy source 21. The switch may be associated with the elongate member 4, e.g., the handle 7, or may be operatively coupled to the elongate member 4, such as a foot switch, to selectively activate the fluid control system 3. In some aspects, the activation element 32 comprises a movable mechanical component, such as a switch or actuator, configured to open a valve to allow fluid to be transported through the one or more fluid paths 23. For example, the activation element 32 may include a switch or actuator operatively coupled to a piston or plunger that may be driven within or against a supply component 30 or fluid path element 23. Pressure resulting from movement of the piston or plunger may induce fluid transport, thus, operating as a transport component 31 to push or pull fluid through the one or more fluid paths 23. In some aspects, the handle 7 includes a switch or actuator, which may be associated with the switch 20a or trigger 20c, that is coupled to the energy source 21 or valve to activate transport of fluid through the one or more fluid paths 23. In various aspects, the activation element 32 may be configured to open a proximal fluid port 24 or a distal fluid port 25. The power may be manual or electrical, e.g., activation of the energy source 21 to provide energy to the end effector 13 may further activate the fluid control system 3. In some aspects, the transport component 31 may, for example, comprise a bulb that may be squeezed to evacuate fluid from within the bulb or to expel or evacuation another fluid through one or more fluid paths 23. In various aspects, the activation element 32 may be coupled to a valve fluidically coupled to the supply component 30 or the fluid path element 23. The activation element 32 may be configured to selectively operate the valve via an electrical or manual switch such that the valve may be opened or closed to control movement of fluid between the outlets of the valve.

Referring to FIG. 1D, an example of further details of the evacuation and irrigation mechanisms of the medical device 2 is depicted. The example fluid pathways and connections may be consistent with the block diagram descriptions in FIG. 1C. Here, an inner fluid tube 130 within the shaft 10 is coupled at the proximal end at direct connection 120 to a fluid manifold 50. The fluid manifold 50 may include a fluid extraction port 115 and a fluid intake port 125, although in this profile view only one of the ports are depicted. The fluid extraction port 115 may allow for evacuation of fluids being evacuationed out of a surgical site from the end effector at the distal end of the shaft 10, while the fluid intake port 125 may allow for transmission of fluids to be applied to the surgical site through the shaft 10 and to the end of the end effector at the distal end of the shaft 10. Also depicted are an evacuation activation button 110 and an irrigation activation button 105. Again, only one button is depicted due to the profile view of this figure. For reference, the grasping trigger 20c, the energy activation button 20a, and the rotation knob 20b are also depicted, to provide an example of how the evacuation and irrigation mechanisms may interact with the additional features of the surgical device 2.

Referring to FIG. 1E, a perspective view of the semi transparent view in FIG. 1D is depicted. In this view, both the fluid extraction port 115 and fluid intake port 125 are clearly illustrated. These ports may connect to hoses or other valves to supply and extract fluid through the surgical device 2. Also depicted are the irrigation activation button 105 and the evacuation activation button 110. The buttons 105 and 110 may be spring biased and coupled to rotating valves within the fluid manifold 50. When un-pressed, the rotating valves within the fluid manifold 50 may be rotated to block passageway through the fluid manifold 50 between the shaft 10 and the ports 115 and 125. Then, when one of the buttons 105 or 110 are pressed, the rotating valves associated therewith may rotate 90° to complete fluid passage between the respective port 125 or 115 to the shaft 110. In some aspects, a latching mechanism coupled between the buttons 105 and 110 and is the fluid manifold 50 may be configured to latch the buttons 105 and 110 into a stable or fixed position to allow continual irrigation or evacuation, respectively, during a surgical procedure. In this way, a user may press down on one of the buttons 105 or 110 to latch said button into place so as to not have to continually press on the button or to maintain the irrigation or evacuation functionality. To unlatch, in some aspects, the user may then press down again on the latch to button, and the associated spring may then extend the button. Other example implementations for providing a latching and unlatching mechanism known to those with skill in the art are possible, and aspects are not so limited.

Referring to FIG. 1F, further example details of the interconnection between the fluid tube and the grasping jaws at the end effector 14 are depicted. In some aspects, a fluid tube 130 is positioned within the shaft 10 and on the inner side of actuation tubes that connect to one or more of the jaws 15a and/or 15b. While it is mentioned that the end effector 14 may be configured to rotate upon rotation of the knob 20b, the inner fluid tube 130 may be configured to not rotate at the same time. For example, the inner fluid tube 130 may be spaced within the shaft 10 and away from the actuation tubes guiding the jaws 15a and/or 15b so as to not touch during rotation. In other cases, the fluid inner tube 130 may comprise a low friction insulation, film, or other material to allow smooth rotation around it and to minimize disruption of the inner tube 130 during said rotation. In other cases, a rotating valve may be included around the fluid inner tube 130 and coupled to the fluid manifold 50, for example, to allow rotation of the inner tube 130 during rotation of the rest of the shaft 10. Also depicted are electrical shorts 135 to help electrically isolate the jaws 15a and 15b.

Referring to FIG. 1G, an example illustration of evacuation and irrigation tubes 150 and 155, as well as a power cable 160, is depicted. The ports described in the previous figures provide examples of how the illustrated tubes and cables may be coupled to the surgical instrument 2. The example power cable 160 may supply wire to power to the surgical instrument 2, while in other cases, the surgical instrument 2 may be powered internally, such as through the use of batteries. Example power systems coupled to the power cable 160 are described in FIG. 49, for example. The evacuation and irrigation tubes 150 and 155 may be configured to be connected to other extension cables or valves.

The following descriptions and related figures provide examples of more detailed designs of the end effector 14, including one or more members for grasping and applying sealing energy, and one or more members with a fluid path for evacuation and irrigation. The following are merely examples, and it may be apparent to those with skill in the art how the various examples may be combined or interchanged to be included in various other aspects, and aspects are not so limited.

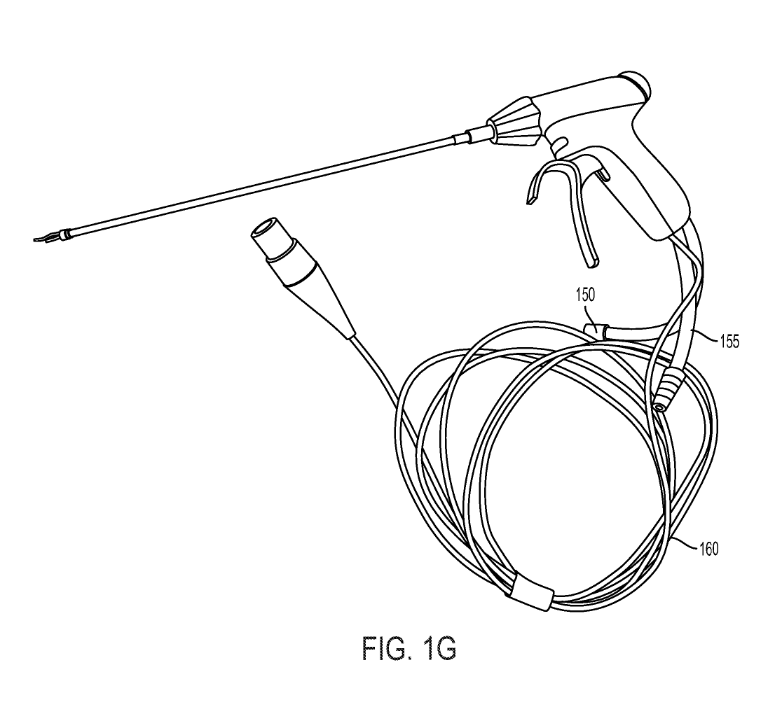

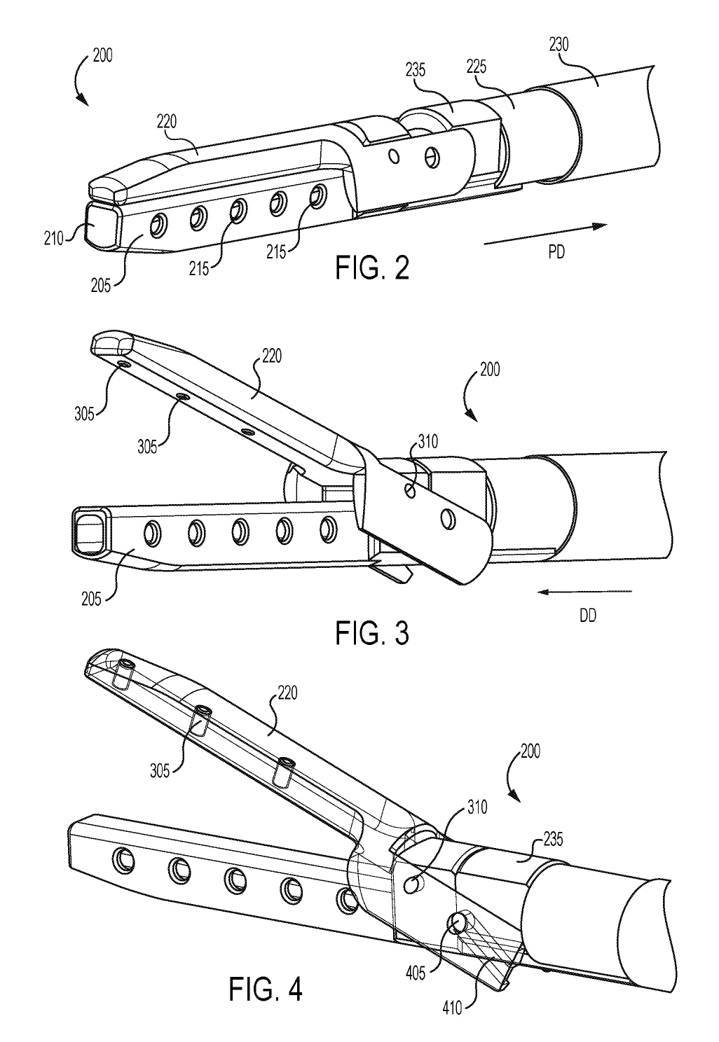

Referring to FIG. 2, illustration 200 depicts one example design of an end effector including jaw members for grasping and applying sealing energy and according to some aspects. Here, a bottom jaw 205 may interact with a top jaw 220 to grasp tissue. The bottom jaw 205 also may include an evacuation and irrigation path that opens or ends principally from the distal end 210 of the bottom jaw 205 and runs through a tubing system through the shaft 10, not depicted. In some aspects, the bottom jaw 205 also may include one or more evacuation and irrigation holes 215 located on the lateral sides of the bottom jaw 205. The holes 215 may allow for additional evacuation and irrigation to occur on the sides of the end effector.

In some aspects, the top jaw 220 and the bottom jaw 205 may be configured to supply electrosurgical energy, such as RF energy, to tissue at a surgical site. The end effector may be configured to supply monopolar electrosurgical energy, in that both jaws 220 and 205 supply energy at a first pole. In other cases, the end effector may be configured in a bipolar arrangement, such that the jaw 205 may supply RF energy at a first pole, while the other jaw 220 may be configured to supply RF energy at a second pole.

As depicted, the end effector also may include a closure saddle 235, a closure tube 225, and a shrink tube 230 and may be used for insulation. In illustration 200, the jaws 205 and 220 are depicted in a closed position, which may be achieved by translation of the closure saddle 235 moved back in the proximal direction, as indicated by the arrow PD.

Referring to FIG. 3, the end effector of the example illustration 200 is now depicted in an open position, where the closure saddle has been translated in the distal direction toward the end of the end effector, as indicated by the arrow DD. Also depicted are insulated pins 305, in this case coupled to the top jaw 220. The insulated pins 305 may provide separation and insulation between the top jaw 220 and the bottom jaw 205, such that only the insulated pins 305 touch the bottom jaw 205 when the jaws are in a closed position. In this way, the top jaw 220 and the bottom jaw 205 may be configured to supply RF energy at different polarities, due to the insulated pins 305 physically separating the jaws 205 and 220 even in the closed position. Also depicted is movement by the top jaw 220 at the pivot pin 310.

Referring to FIG. 4, the example end effector of illustration 200 is depicted with a semi transparent view, in order to depict some further details of performing opening and closing of the jaws, according to some aspects. As depicted, while the top jaw 220 pivots based on the pivot pin 310, movement is driven by a cam 405 connected to the closure saddle 235 and movable within closure slot 410. That is, while the closure saddle 235 is translated along the shaft 10 in the distal direction DD, the cam 405 slides within the closure slot 410 to the distal end of the closure slot 410. Due to the lower position of the cam 405 relative to the pivot pin 310, this motion causes the top jaw 222 pivot upward to the open position. Conversely, as the closure saddle 235 is translated along the shaft 210 and the proximal direction PD, the cam 405 slides back within the closure slot 410 to cause movement of the top jaw 220 to rotate to the closed position.

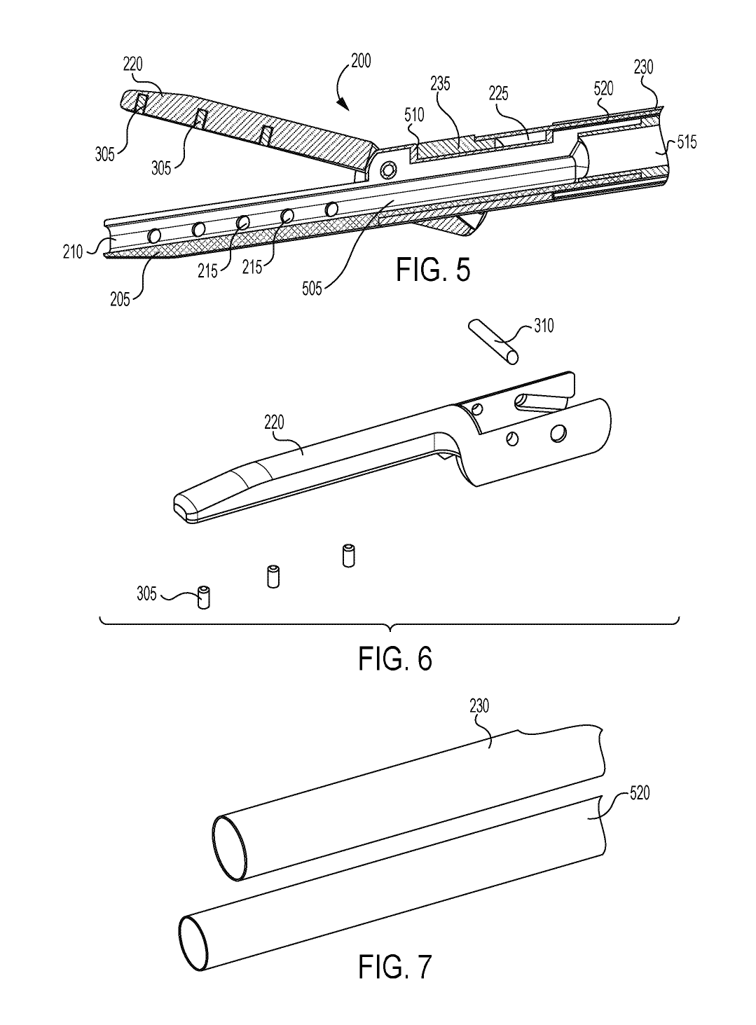

Referring to FIG. 5, a longitudinal cross-sectional view of the end effector in illustration 200 is depicted to provide illustration of additional detail, according to some aspects. For example, a longitudinal cross-sectional cutout of the bottom jaw 205 is depicted to reveal the irrigation path 505 running the length of the bottom jaw 205, including the evacuation and irrigation holes 215 and ending at the distal end 210. The proximal end of the irrigation path 505 connects to an irrigation ground tube 515. Also depicted is the top jaw 220 with the insulated pins 305 substantially embedded into the top jaw 220. Various layers of insulation are also depicted, such as insulating layer 510, an insulating tube 520 insulating the irrigation ground tube 515, and the shrink tube 230 providing insulation over the entire contents of the shaft 10. Also depicted are the closure tube 225 and the closure saddle 235.

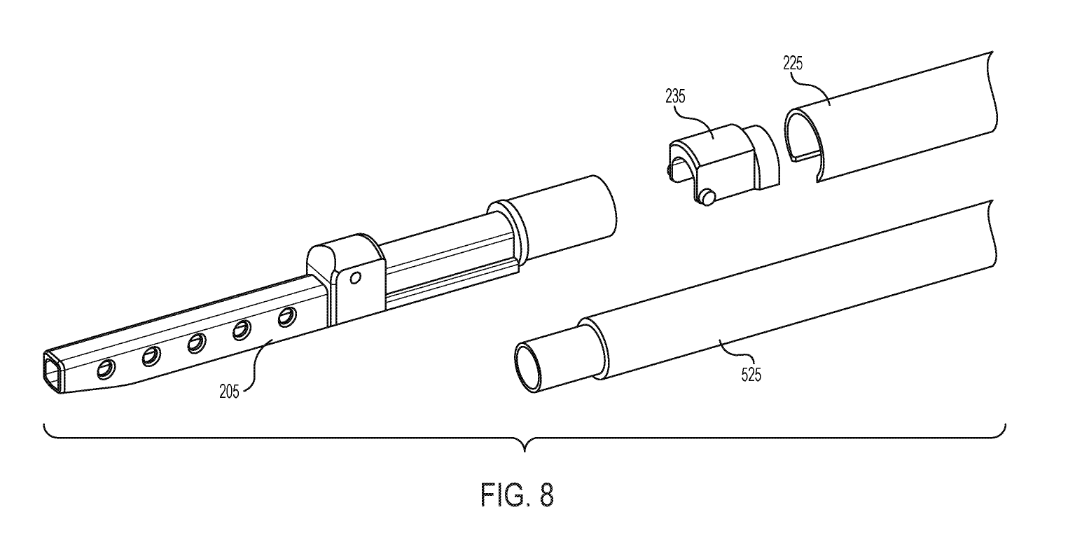

Referring to FIGS. 6-8, the end effector of illustration 200 is depicted in various exploded views to isolate the individual parts. In FIG. 6, the top jaw 220 is depicted with the insulated pins 305 and the pivot pin 310 being separated. In FIG. 7, examples are depicted of the shrink tube 230 and the insulating tube 520. In FIG. 8, example components are depicted of the bottom jaw 205, the closure saddle 235, the closure tube 225, and the irrigation ground tube 525. It may be apparent to those with skill in the art how the various components in these exploded views may be assembled to complete the end effector of the example illustration 200.

Mechanical Advantage Grip Alternative Aspect

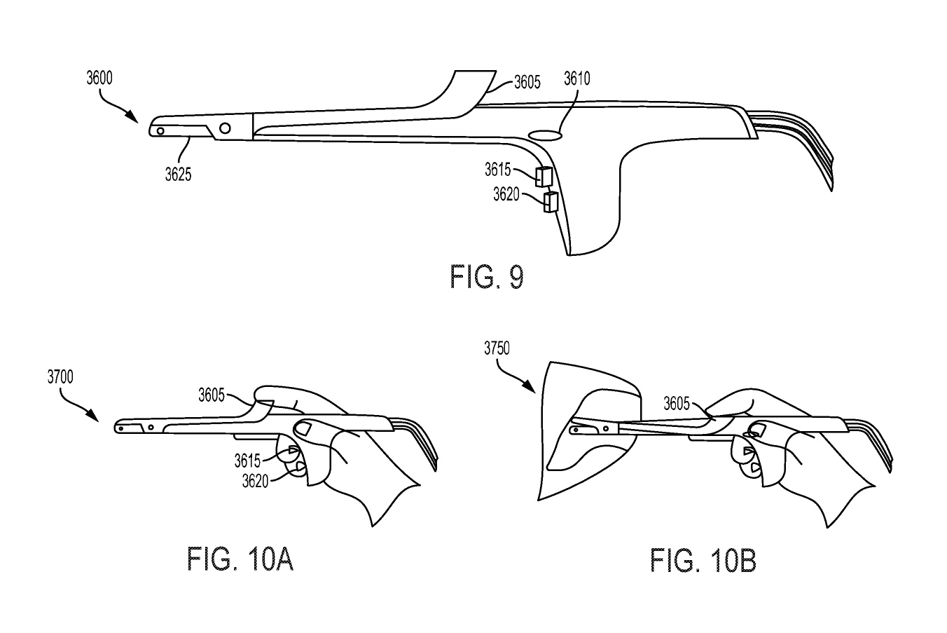

Referring to FIG. 9, illustration 3600 depicts another example design of a medical instrument with bipolar jaws on the end of an evacuation wand. In surgery, blood from cut or nicked vessels can obscure the visual fields of the vessel that was damaged, making it difficult for a surgeon to seal the vessel. Typically, an evacuation device is used to remove the blood and then another device is used to seal the vessels. However, once the evacuation is moved to make way for the sealing device, the blood flows again, and it is difficult to see the so again. A solution is needed to allow the doctor to evacuation and seal at the same time. In certain cases, a medical device in the shape of tweezers or being used in a pencil grip may be a solution that offers more precise control to resolve these issues.

Illustration 3600 is an example of such a device that includes bipolar jaws and an evacuation mechanism at the end effector 3625. The grasping lever 3605 can allow the jaws to open and close through the use of a curling iron like lever arm, with the arm is held in a closed position by spring. The surgeon may hold the device in a pencil grip like fashion. The spring force holding the jaws closed may be sufficient enough to let the jaw shut with enough load to ensure that the tissue is compressed enough to result in a good sealing. In addition, when opening the jaws, it may be useful to reduce the load so that holding the jaws open does not fatigue the user. A particular mechanical mechanism is included in this design to resolve this issue. In general, the grasping lever 3605 is coupled to a spring lever and mechanism that starts with a high mechanical advantage from the spring to put load on the closed jaws and ends with a high mechanical advantage from the finger to the spring when the jaws are open. As depicted, the medical device also includes a bipolar controller button 3610, irrigation control 3615 and evacuation control 3620.

Referring to FIGS. 10A and 10B, the example illustrations 3700 and 3750 depict how the device in illustration 3600 may be used. As depicted, a user may hold the device as one might grip a pencil, with the index finger being used to manipulate the grasping lever 3605. The ring finger may be used to control the irrigation control button 3615, while the pinky finger may be used to control the evacuation button 3620. The evacuation and irrigation controls may be utilized when the device has the jaws in the closed position, which is depicted in illustration 3700. As depicted in illustration 3750, the user may press down on the grasping lever 3605 to open the jaws for grasping. The jaws may then be closed by the user releasing the grasping lever 3605, reverting back to the position in illustration 3700. Once in this position, the user may press on the bipolar control 3610 with the thumb to activate bipolar sealing.

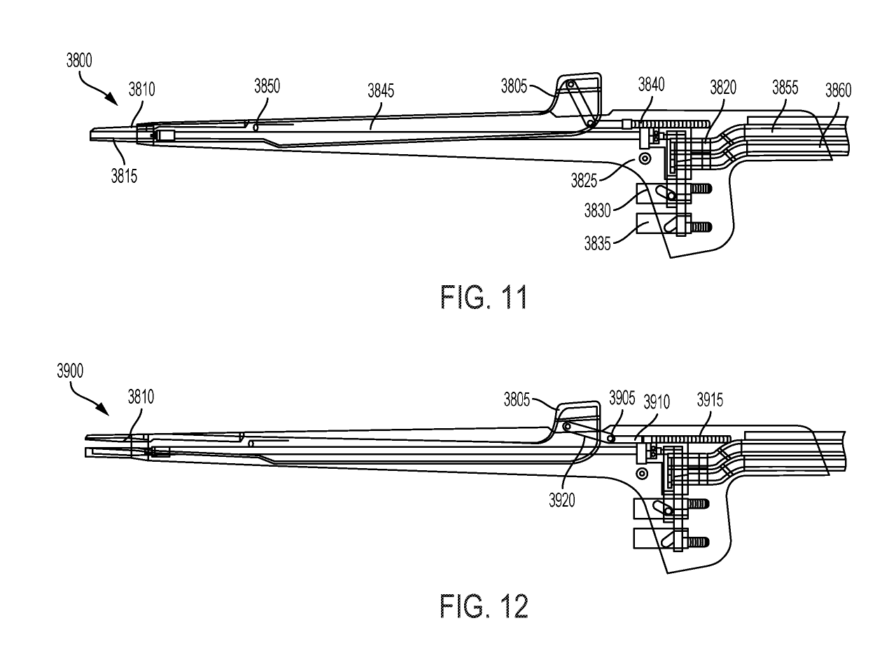

Referring to FIG. 11, illustration 3800 provides a view of some of the inner workings of the pencil grip irrigation and evacuation medical device grasper, as introduced in FIG. 9, according to some aspects. Here, the clamp arm 3805 includes a joint about a pivot that is mechanically coupled to a spring torsion system 3840. The other end of the lever component 3845 that is connected to the clamp arm 3805 includes the upper jaw 3810. The lever component 3845 may pivot about a joint 3850. The lower jaw 3815 includes an evacuation component that will be depicted in closer detail in a following figure. Also depicted is a valve manifold assembly 3820, including tubes 3855 and 3860 for irrigation and evacuation, respectively. The valves may be controlled by the irrigation button 3830 and the evacuation button 3835, respectively. Also depicted is the RF energy switch 3825 that is connected to the button on the outside.

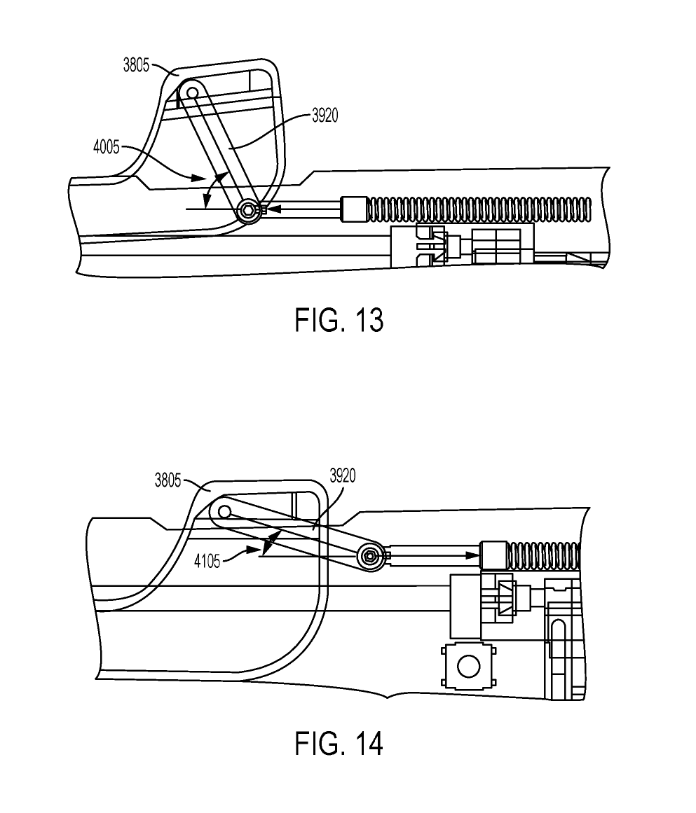

Referring to FIG. 12, illustration 3900 depicts a change in some of the mechanical movements when the clamp arm 3805 is pressed down. As depicted, the linkage at the joint 3905 causes the horizontal bar 3910 to compress the spring 3915. On the other end, bar 3920 connected to the joint 3905 and the pivot joint in the clamp arm 3805 is configured to change angles from a more vertical position to a more horizontal position when the clamp arm 3805 is pressed down. This creates a mechanical advantage for the user's finger when the clamp arm 3805 is pressed down, and conversely creates mechanical advantage for the spring 3915 when the clamp arm is not pressed. In this way, higher mechanical advantage to the spring 3915 allows the upper jaw to remain more strongly clamped down when the clamp arm 3805 is not pressed, while higher mechanical advantage to the users finger allows the upper jaw to remain open more strongly when the clamp arm 3805 is pressed down. FIGS. 13 and 14 provide a closer view of these principles, highlighting how the change in angle (see angle 4005 vs. angle 4105) of the mechanical bar 3920 transfers the mechanical advantage appropriately, based on if the clamp arm 3805 is pressed down or not. In general, to create the transfer of mechanical advantage according to some of the disclosures herein, when the clamp arm 3805 is not pressed down, the lever arm 3920 should have an angle of incidence 4005 greater than 45° and no more than 90°, while the lever arm 3920 should have an angle of incidence 4105 less than 45° but more than 0° when the clamp arm 3805 is pressed down.

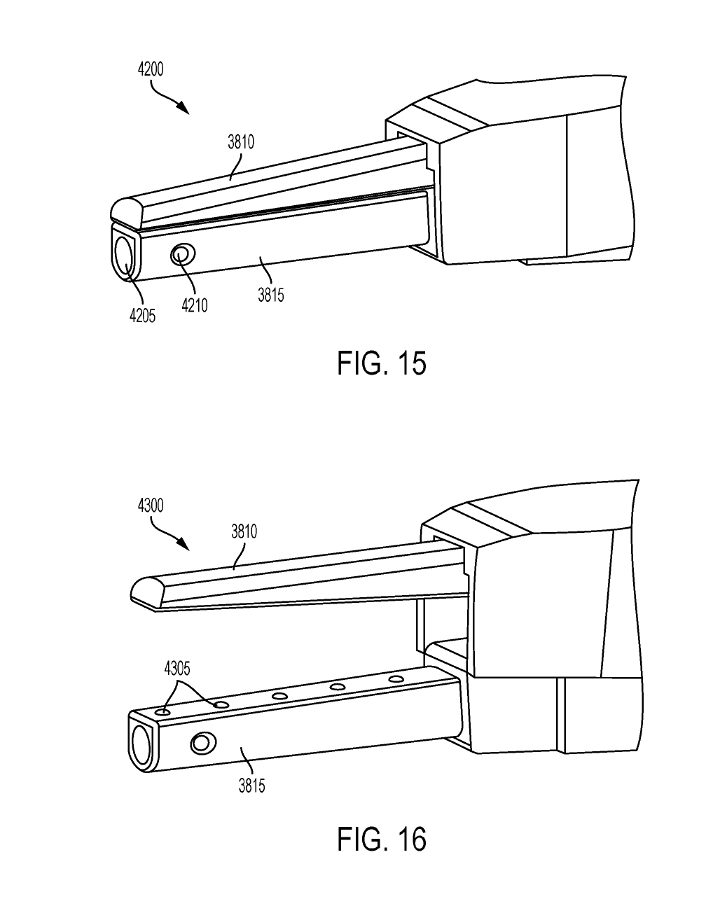

Referring to FIG. 15, illustration 4200 depicts a closer view of the distal end of the medical device with the pencil grip, according to some aspects. As depicted, the top jaw 3810 is closed on the lower jaw 3815. The lower jaw 3815 also includes a channel 4205 for evacuation and irrigation that is fluidically coupled via a shaft to the out manifold assembly 3820 (see FIG. 11). In some cases, one or more side channels 4210 may also be present in the sides of the lower jaw 3815, as depicted. In FIG. 16, illustration 4300 depicts how the upper jaw 3810 and lower jaw 3815 may appear when the jaws are in an open position. Because one or more of the jaws also is configured to transmit electrosurgical energy, and because the jaws may be configured at different polarities, insulating pins 4305 may be present in one or more of the jaws to electrically isolate one jaw from another.

Closed Loop Modulated Vacuum System

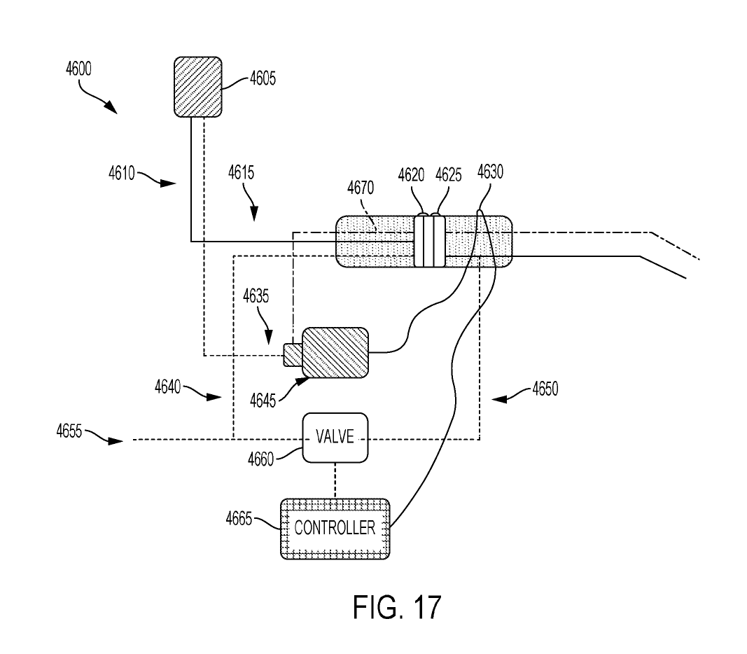

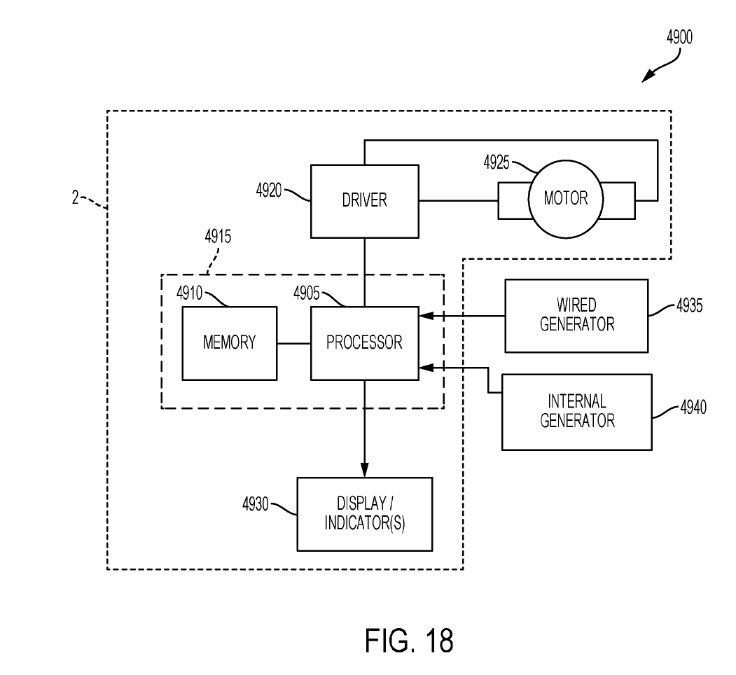

Referring to FIGS. 17 and 18, in some aspects, a closed-loop modulated vacuum system may also be included to control evacuation and irrigation at a surgical site. Irrigation may be used for rinsing and cleaning purposes, such as to clear away debris and to provide sufficient rinsing of some areas. In some cases, saline is used as the liquid agent, and may sometimes be featured in a saline drip at the surgical site. Conventionally, some devices may allow saline to flow over the tissue being coagulated at different flow rates set by a user on a generator. However, extra saline can burn on intended tissue or even the surgeon's hand unexpectedly. To mitigate this, typically a surgeon utilizes an external evacuation tube near the electrodes to remove the extra saline during the procedure with a second hand or with an assistant. In general, the precise amount of saline or other irrigation liquid is important to prevent unintended burning and allow for proper rinsing and coagulation.

The following figures and descriptions therefore introduce a method to control a vacuum or evacuation process. Too much vacuum may not allow the intended tissue to coagulate, which then allows the tissue to dry out too quickly and causes the electrodes to stick to the tissue. Too little vacuum tends to leave extra saline or other liquid unattended at the tissue surface, which can then lead to unintended extra surface burning. The following figures and description present a pulse or modulation of the vacuum or evacuation to trigger at specific intervals or frequencies, depending on the power to the electrodes being delivered, thereby allowing the saline or other liquid to coagulate the tissue at a controlled rate while not burning unintended tissues or the surgeon's hands.

Referring to FIG. 17, illustration 4600 provides a block system diagram of a control system involving the fluid irrigation and vacuuming through a medical device, according to some aspects. As depicted, the various components include a saline bag or bag for other fluids 4605, a generator 4645 coupled to a pump 4635, the main components of the medical device 4670, vacuum from the outside, such as a wall 4655, and a valve 4660 coupled to a controller 4665. The saline bag 4605 may have one to 4610 to allow for full irrigation connected directly to the medical device 4670. Another two may be used for dripping that is connected to the pump and generator 4645. From the pump 4635, the dripping tube line 4615 is then coupled to the medical device 4670. The generator 4645 may control the pump 4635, which controls the amount of dripping from the saline bag 4605. In addition, the vacuum 4655 may have two lines: one for full vacuum functionality 4640 directly connected to the medical device 4670, while another is connected to a valve 4662 allow for pulsing vacuum 4650 controlled by the controller 4665. The pulsing vacuum line 4650 may then be directly connected to the medical device 4670. Electrical lines may be connected to both the generator 4645 and the controller 4665 from button one 4630, strives coagulation energy and dripping control functionality. The controller 4665 may control the valve 4640 that is used to determine an amount of evacuation from the vacuum 4655. The other lines 4610, 4615, and 4640 may feed directly into a switch that includes both button two 4625 for full vacuuming, as well as button three 4620 for irrigation. As previously mentioned, an amount of saline or other fluid may be directly proportional to an amount of RF energy used in the coagulation process. Therefore, button one 4630 may be configured to apply various levels of electrosurgical energy, which in turn may proportionally drive an amount of vacuuming by the controller 4665.

In some aspects, when button one 4630 is pressed, the valve 4660 works in pulsing mode controlled by the controller 4665, which handles the pulsing vacuum functionality 4650. In concert, the generator 4645 outputs RF energy while also driving the control of the saline drip from the pump 4635. When button one 4630 is released, the valve 4660 closes based on commands from the controller 4665. The generator 4645 may then also stop providing an RF output, as well as stopping the pump 4635. Separately, pressing the button two 4625 provides for full vacuuming only, while pressing button three 4620 provides for full irrigation only.

In some aspects of the valve, the valve 4660 is normally closed as a safety procedure, and the pulsing of the saline drip and vacuum are activated only when button one 4630 is pressed. In some aspects, the value specifications also include inlet vacuum specifications of 650 mmHg, a flow rate 2 SCFM, frequency of 0.1 at ˜100 Hz, and a duty ratio of 10% to ˜90%.

FIG. 18 is a block diagram of a surgical system 4900 comprising a motor-driven surgical grasping instrument 2 (e.g., FIG. 1) with evacuation and irrigation mechanisms, the surgical instrument coupled to a generator 4935 (4940), according to some aspects. The motor-driven surgical cutting and fastening instrument 2 described in the present disclosure may be coupled to a generator 4935 (4940) configured to supply power to the surgical instrument through external or internal means. While previous figures describe examples of how the irrigation and evacuation mechanisms may be implemented in the surgical instrument 2, FIG. 9 describes examples of the portions for how electrosurgical energy may be delivered to the end effector. In certain instances, the motor-driven surgical instrument 2 may include a microcontroller 4915 coupled to an external wired generator 4935 or internal generator 4940. Either the external generator 4935 or the internal generator 4940 may be coupled to A/C mains or may be battery operated or combinations thereof. The electrical and electronic circuit elements associated with the motor-driven surgical instrument 2 and/or the generator elements 4935, 4940 may be supported by a control circuit board assembly, for example. The microcontroller 4915 may generally comprise a memory 4910 and a microprocessor 4905 (“processor”) operationally coupled to the memory 4910. The processor 4905 may control a motor driver 4920 circuit generally utilized to control the position and velocity of the motor 4925. The motor 4925 may be configured to control transmission of energy to the electrodes at the end effector of the surgical instrument. In certain instances, the processor 4905 can signal the motor driver 4920 to stop and/or disable the motor 4925, as described in greater detail below. In certain instances, the processor 4905 may control a separate motor override circuit which may comprise a motor override switch that can stop and/or disable the motor 4925 during operation of the surgical instrument in response to an override signal from the processor 4905. It should be understood that the term processor as used herein includes any suitable microprocessor, microcontroller, or other basic computing device that incorporates the functions of a computer's central processing unit (CPU) on an integrated circuit or at most a few integrated circuits. The processor is a multipurpose, programmable device that accepts digital data as input, processes it according to instructions stored in its memory, and provides results as output. It is an example of sequential digital logic, as it has internal memory. Processors operate on numbers and symbols represented in the binary numeral system.

In some cases, the processor 4905 may be any single core or multicore processor such as those known under the trade name ARM Cortex by Texas Instruments. In some cases, any of the surgical instruments of the present disclosures may comprise a safety processor such as, for example, a safety microcontroller platform comprising two microcontroller-based families such as TMS570 and RM4x known under the trade name Hercules ARM Cortex R4, also by Texas Instruments. Nevertheless, other suitable substitutes for microcontrollers and safety processor may be employed, without limitation. In one instance, the safety processor may be configured specifically for IEC 61508 and ISO 26262 safety critical applications, among others, to provide advanced integrated safety features while delivering scalable performance, connectivity, and memory options.

In certain instances, the microcontroller 4915 may be an LM 4F230H5QR, available from Texas Instruments, for example. In at least one example, the Texas Instruments LM4F230H5QR is an ARM Cortex-M4F Processor Core comprising on-chip memory 4910 of 256 KB single-cycle flash memory, or other non-volatile memory, up to 40 MHz, a prefetch buffer to improve performance above 40 MHz, a 32 KB single-cycle serial random access memory (SRAM), internal read-only memory (ROM) loaded with StellarisWare® software, 2 KB electrically erasable programmable read-only memory (EEPROM), one or more pulse width modulation (PWM) modules, one or more quadrature encoder inputs (QEI) analog, one or more 12-bit Analog-to-Digital Converters (ADC) with 12 analog input channels, among other features that are readily available for the product datasheet. Other microcontrollers may be readily substituted for use in the motor-driven surgical instrument 2. Accordingly, the present disclosure should not be limited in this context.

Referring again to FIG. 18, the surgical system 4900 may include a wired generator 4935, for example. In certain instances, the wired generator 4935 may be configured to supply power through external means, such as through electrical wire coupled to an external generator. In some cases, the surgical system 4900 also may include or alternatively include an internal generator 4940. The internal generator 4940 may be configured to supply power through internal means, such as through battery power or other stored capacitive source. Further descriptions of the internal generator 4940 and the wired generator 4935 are described below.

In certain instances, the motor-driven surgical instrument 2 may comprise one or more embedded applications implemented as firmware, software, hardware, or any combination thereof. In certain instances, the motor-driven surgical instrument 2 may comprise various executable modules such as software, programs, data, drivers, and/or application program interfaces (APIs), for example.

Bipolar Coagulation Electrode with Field Evacuation and Irrigation

The following figures and descriptions introduce a surgical device configured with a full integration of evacuation and irrigation functions. In some open surgeries, such as some solid organ surgeries, particularly open liver surgeries, hemostasis and visualization are a considerable challenge for surgeons. In solid organ procedures, the tissue is fragile and prone to bleed. Keeping a dry field and maintaining good visualization is a key need in open procedures. It is difficult to control the large amount of bleeding in these procedures. The use of currently available devices can be problematic due to inefficiencies of instrument exchange, inadequate hemostasis of parenchymous tissue, and device-specific use complexities. Incorporation of evacuation and irrigation into a bipolar device could create a multifunctional instrument that assists surgeons in achieving hemostasis and visualization during open solid organ surgeries. This surgical device may provide large area, soft bipolar coagulation with no char. This surgical device may also provide non-stick energy delivery by way of irrigation. Further, this device can provide controlled fluid delivery and removal (evacuation) providing minimal user management of the surgical field by fluid circuit formed by separate irrigation and evacuation lumen. This device may also be able to temporarily stop bleeding by en-face compression during energy delivery resulting in fast coagulation/sealing of active bleeding sights—blunt electrode tip with flush to slightly proud electrodes. When using this device, the user can visualize the surgical field and identify precise location of bleeding sources because of the large evacuation lumen for large fluid volume evacuation and evacuation of mist, smoke, plume, etc. The device also provides irrigation for clearing debris and localizing bleeding sources. Bipolar energy is better suited to fragile tissue such as liver parenchyma. Further, bipolar technology is flexible, and it is possible to develop a variety of tip geometries that would not be possible with other modalities (harmonic).

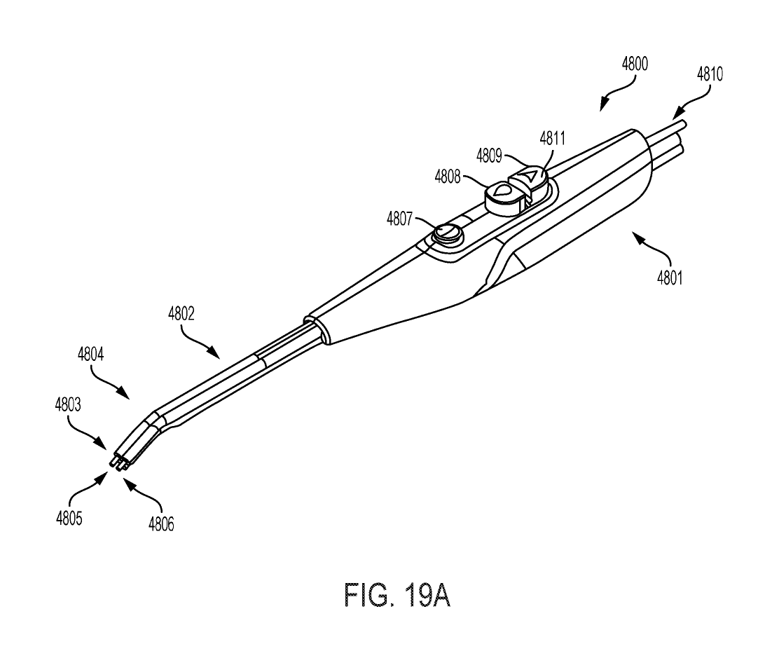

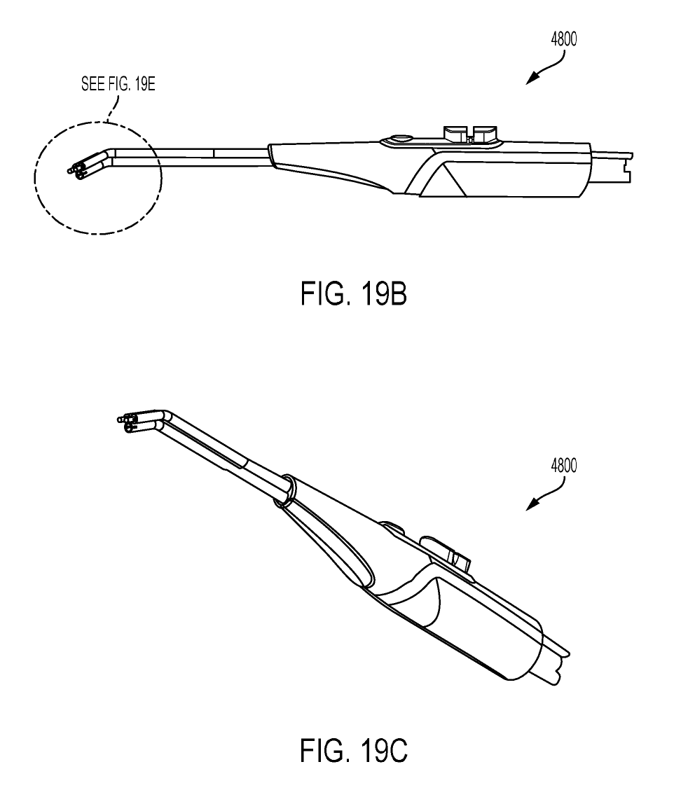

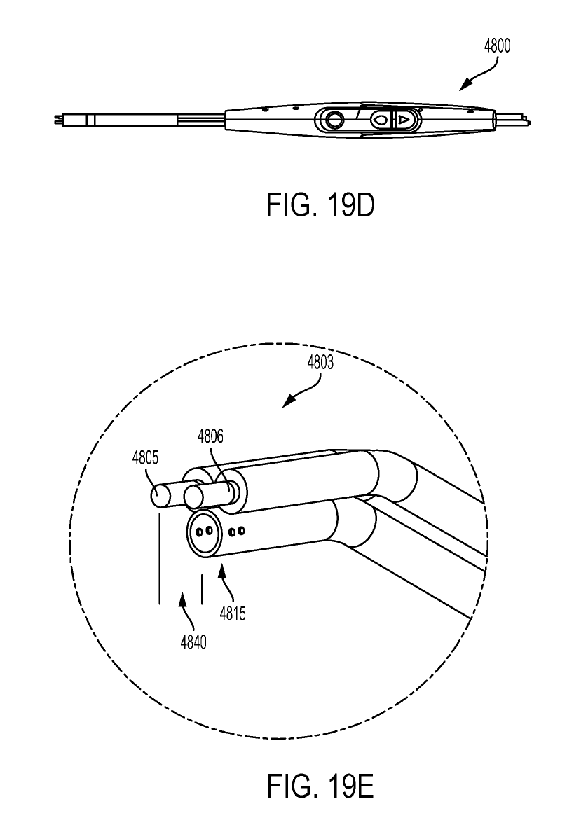

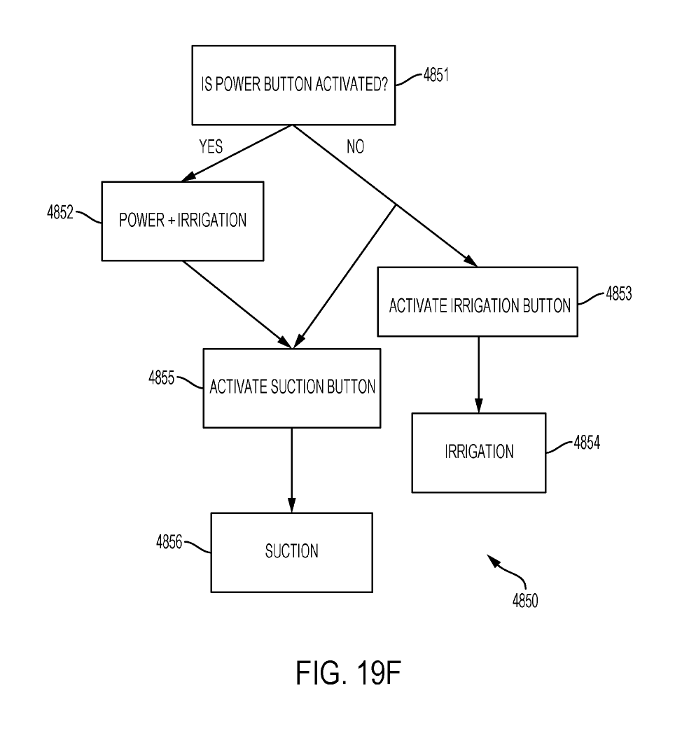

FIGS. 19A-19E depict different views of a surgical device 4800 with a full integration of evacuation and irrigation functions. The surgical device 4800 may include a handle 4801, an elongate member 4802 extending from the handle 4801, and an end effector 4803 at the distal end 4804 of the elongate member 4802. Two electrodes 4805 and 4806 of the opposite polarities may be provided at the end effector 4803. The end effector 4803 may also include an irrigation lumen and an evacuation lumen 4815. An irrigation tube may extend from the irrigation lumen along the elongate member 4802 to the handle 4801. An evacuation tube may extend from the evacuation lumen 4815 along the elongate member 4802 to the handle 4801.

It may be recognized that the coordination of the functions of the electrodes 4805,4806, the irrigation lumen, and the evacuation lumen 4815 is necessary for effective use of a surgical device (for example, surgical device 4800). If the end of the evacuation lumen 4815 is located too far in a proximal direction (towards the user), fluid from the irrigation lumen may build up in the surgical site thereby obscuring the user's view. However, if the evacuation lumen 4815 is located too far in a distal direction (away from the user), fluid from the irrigation lumen may be evacuated prematurely, thereby obviating the ability of the fluid to clean the surgical site. Therefore, an effective distance 4840 between the distal end of the electrodes 4805,4806 and the distal end of the evacuation lumen 4815 may be critical for effective cleaning and debriding of the surgical site. It has been determined that such effective distance 4840 between the distal end of the electrodes 4805,4806 and the distal end of the evacuation lumen 4815 may encompass a range of about 0.1 inch to about 0.5 inches, in which the distal end of the evacuation lumen 4815 is located proximally to the distal end of the electrodes 4805,4806. Examples of the effective distance 4840 may include, without limitation, about 0.1 inch, about 0.2 inches, about 0.3 inches, about 0.4 inches, about 05 inches, and any value or range of values therebetween, including endpoints.

The handle may include a power button 4807, an irrigation button 4808, and an evacuation button 4809. The irrigation button 4808 may have an embossed symbol that represents water to depict that the button 4808 activates irrigation. The evacuation button 4808 may have an embossed arrow pointing proximally toward the cable end 4810 of the surgical device 4800.

When the power button 4807 with suitable electrical components is pressed down, an electric circuit may be closed, and bipolar energy for coagulation may be provided to the end effector 4803. The power button 4807 can also be activated mechanically by providing proper mechanical linkage. At the same time, the irrigation function may also be activated, and fluid may be provided through the irrigation tube to the irrigation lumen at the end effector 4803. The evacuation function may be activated manually by the user pressing the evacuation button 4808. The user may manually activate evacuation when a drier field or more coagulation effect is desired, for example, when the user wants to evacuate fluids for improved visibility or wants deeper, darker thermal treatment of the tissue. Even when the power button 4807 is not activated, the user may still get evacuation and/or irrigation by pressing the appropriate evacuation button 4809 or irrigation button 4808 or both buttons. This manual approach may be preferred by the user since it gives the user great control of thermal effect and allows bleeding sites, particularly active bleeding from large vessels, to be coagulated. Larger vessels can be addressed better with evacuation because the evacuation helps dry the field by removing bleeding from the site and maximize thermal effect.

In some aspects, the user needs to keep pressing the evacuation button 4809 to keep evacuation activated. In some other aspects, the evacuation button 4809 may be latchable. When the evacuation button 4809 is pressed the first time, the button 4809 stays pressed, and evacuation stays activated. When the button 4809 is pressed again, the button is released from its pressed position, and evacuation is deactivated. In these cases, the user does not have to keep pressing the evacuation button 4809 to keep evacuation activated, so that the user does not need to worry about evacuation and can focus on the surgery. The evacuation button 4809 may be latchable by an undercut in the button 4809 that engages a ledge in the housing of the handle 4801. The ledge may be closed with an activation force that is not perpendicular to the button's finger engaging surface 4811.

As depicted in the flow chart 4850 of FIG. 19F, as described above, when the power button is activated 4851, both power and irrigation are provided 4852. The user can activate the evacuation button 4855 to get evacuation 4856. When the power button is not activated 4851, the user can activate the irrigation button 4853 to get irrigation 4854 and/or activate the evacuation button 4855 to get evacuation 4856.

In some instances, when the power button 4807 is not activated, automatic, continuous or pulsed evacuation may be provided, and when the power button 4807 is activated, irrigation may be turned on with the energy for coagulation, and evacuation may be turned off.

In other instances, evacuation may be provided based on the impedance of the tissue. When the field is dry, impedance is high, so evacuation may be lowered or even shut off immediately. Irrigation may be conversely turned on, or irrigation flow rate may be turned up. When the field is wet, impedance is relatively low, so evacuation may be turned on or raised if low impedance is maintained for a certain time. Irrigation may be conversely turned off, or irrigation flow rate may be turned down. At least one of the manual approach, automatic evacuation, and impedance-based evacuation may be provided in the surgical device 4800.

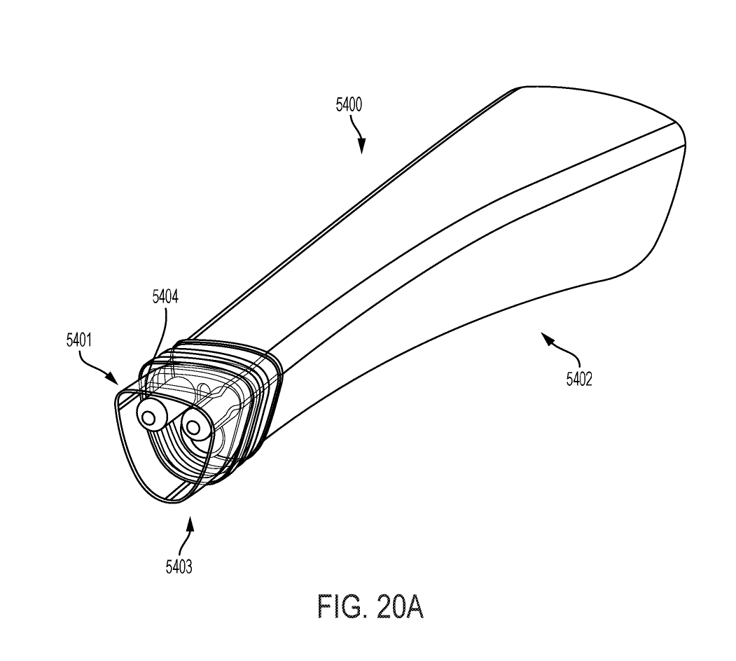

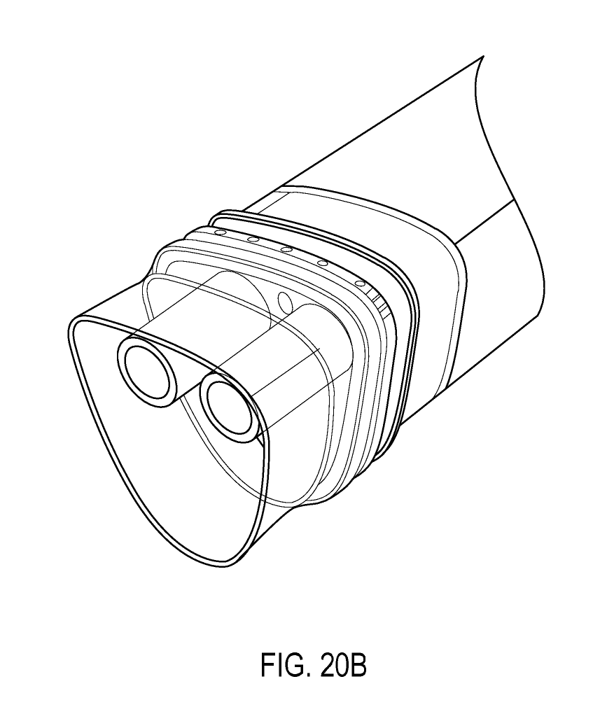

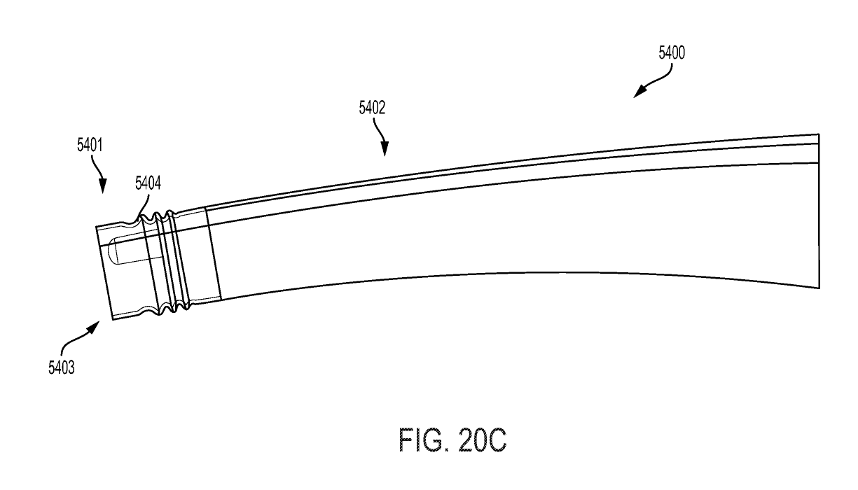

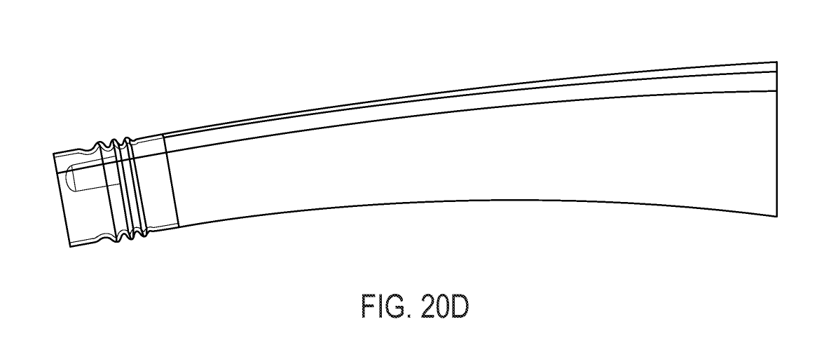

FIG. 20A depicts the distal end 5401 of another aspect of the surgical device with a full integration of irrigation and evacuation. The end effector 5403 is provided on the distal end 5401 of the elongate body 5402 of the surgical device. A boot 5404 may be provided at the distal end 5401 around the end effector 5403 including the two electrodes, irrigation lumen, and evacuation lumen. The boot 5404 may be optically transparent or semi-transparent and made of compliant plastic or elastomeric material. The boot 5404 can capture or contain irrigation fluid at the electrodes or the tip of the surgical device. FIG. 20B is another illustration of the distal end 5401 as depicted in FIG. 20A. FIG. 20C depicts a side view of the distal end 5401 as depicted in FIG. 20A. FIG. 20D is another illustration of the distal end 5401 as depicted in FIG. 20C.

FIG. 21A depicts the distal end 5501 of another aspect of the surgical device with a boot 5502 covering the end effector 5503 at the distal end 5501. As depicted in FIG. 21A, the end effector 5503 includes two electrodes 5504-5505 of opposite polarity, an irrigation lumen 5506, and an evacuation lumen 5507. The irrigation lumen 5506 may be provided between the two electrodes 5507. FIG. 21B is another illustration of the distal end 5501 depicted in FIG. 21A.

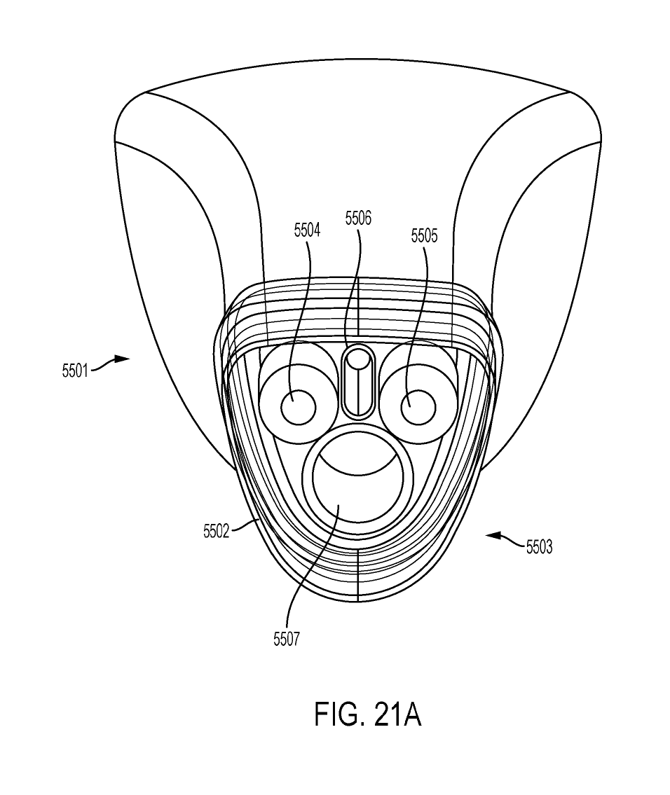

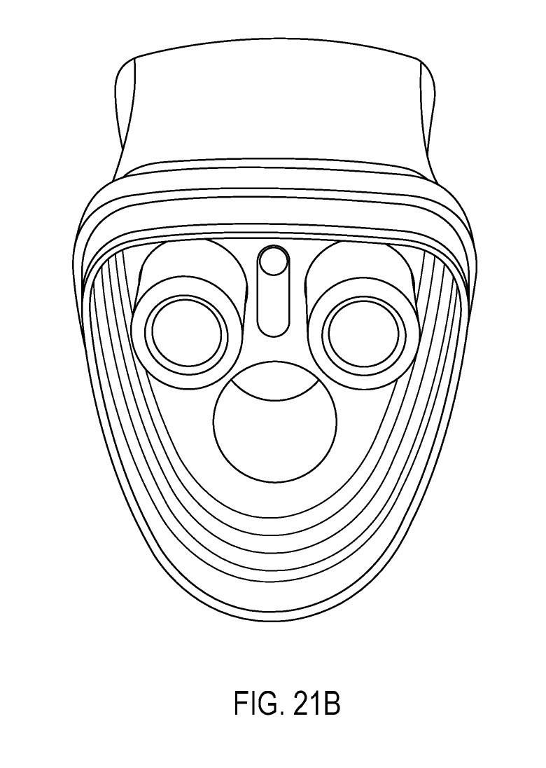

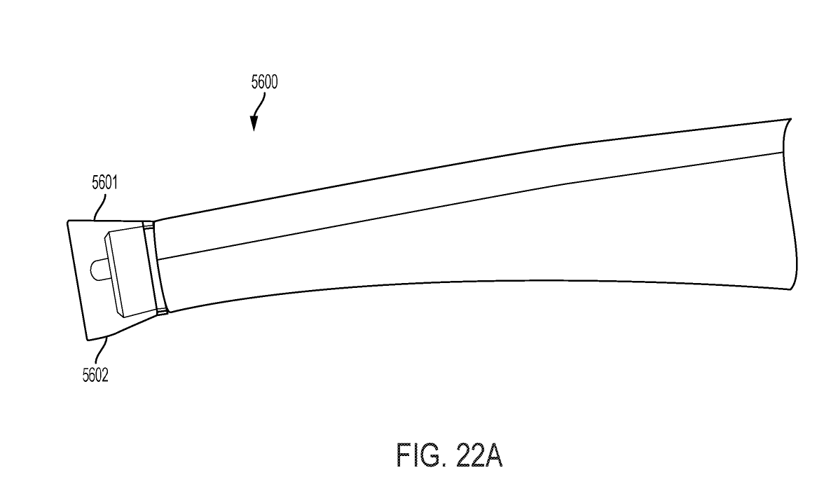

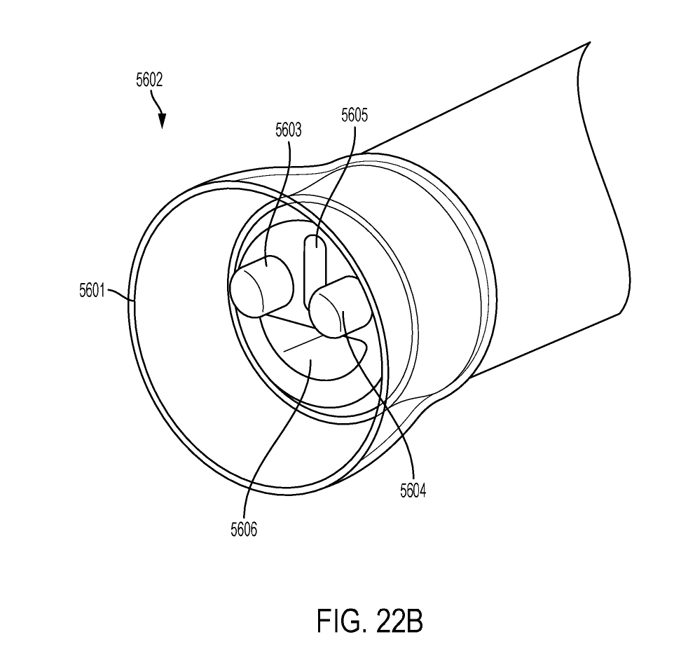

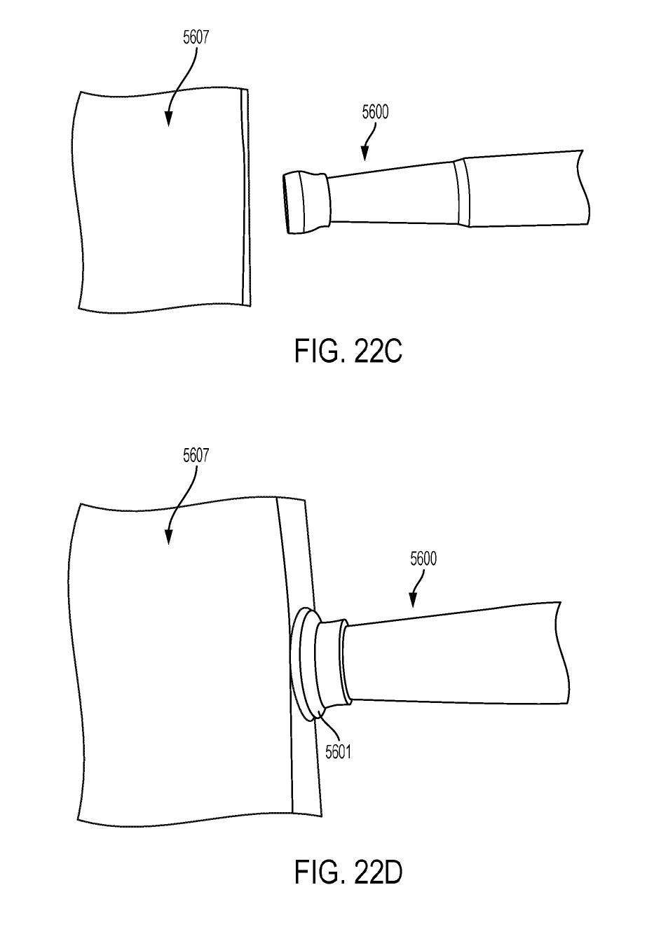

The boot can have any shape depending on the shape of the tip of the distal end of the surgical device. FIGS. 22A,B illustrate another aspect of the boot 5601 covering the end effector 5602 at the distal end 5600 of the surgical device. As depicted in FIGS. 22A,B, the distal end 5600 has a round surface at the tip on which the two electrodes 5603-5604, irrigation lumen 5605, and evacuation lumen 5606 are provided. The irrigation lumen 5605 may be provided between the two electrodes 5603-5604. The evacuation lumen 5606 may be provided below the two electrodes. The boot 5601 is sized and configured to fit the tip of the distal end 5600. The boot 5601 may be transparent or semi-transparent and compliant.

FIG. 22C is another illustration of the distal end 5600 with the boot 5601 before it touches the mock tissue. FIG. 22D depicts the distal end 5600 touching the mock tissue 5607. When the distal end 5600 touches the tissue 5607, the compliant boot 5601 changes its shape to conform to the tissue surface when pressed against the tissue surface. As depicted in FIGS. 22C,D, the boot 5601 is semi-transparent.

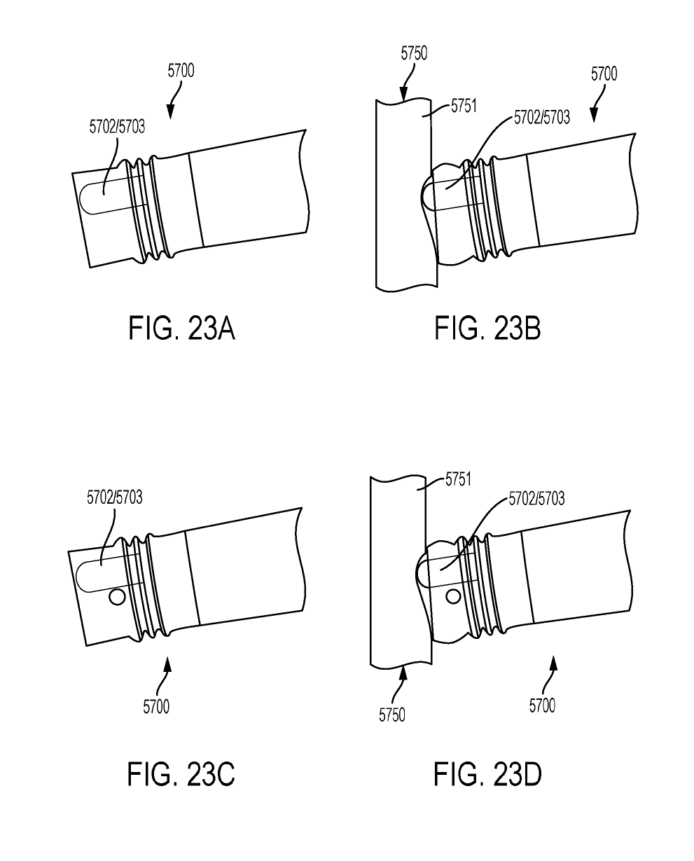

FIGS. 23A-23D illustrate side views of the distal end 5700 with the boot 5701 of the surgical device before and when the distal end 5700 touches tissue 5750. FIGS. 23A and 23C depict the original state of the distal end 5700 before it touches any tissue. FIGS. 23B and 23D depict the distal end 5700 touching the tissue 5750. As depicted in FIGS. 23B and 23D, when the distal end 5700 touches the tissue 5750, the compliant boot 5701 changes its shape depending on the tissue surface 5751, the angle from which the distal end 5700 approaches the tissue surface 5751, and the force applied on the distal end 5700 by the user. When the user keeps applying the force from the distal end 5700 onto the tissue surface 5751, the boot 5701 is pushed back by the tissue surface 5751 to the extent that the electrodes 5702/5703 are exposed outside of the boot 5701. Because FIGS. 23A-23D are side views of the distal end 5700, only one electrode 5702 is depicted here, and the other electrode 5703 is not depicted since it is behind the electrode 5702 in these figures. In some aspects, one or more side ports or vents may be provided on the boot 5701. The one or more side ports or vents can minimize tissue grabbing by the tip of the distal end 5700 because of pressure difference inside and outside of the space surrounded by the tip of the distal end 5700, the inside surface of the boot 5701, and the tissue surface 5751 because of evacuation. The boots 5502, 5601 may also include one or more side ports or vents, which are not depicted in the figures.

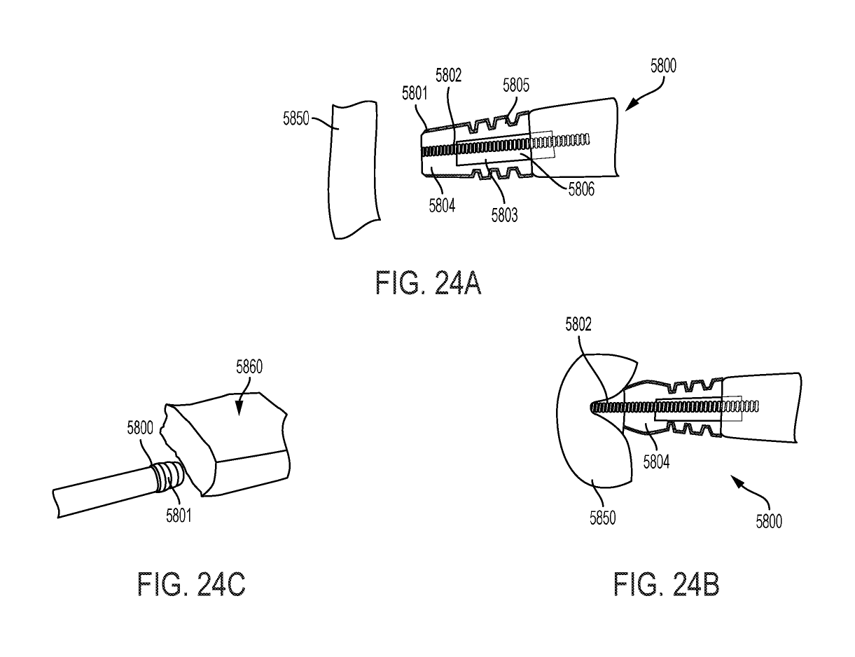

The boot 5801 may also include ribs 5805 such as illustrated in FIGS. 24A-24C. The end effector 5800 may include a hard plastic part 5803 within which the electrodes 5802 and fluid lumen 5806 are provided. The electrodes 5802 extends outside of the hard plastic part 5803. A soft part 5804 with the ribs 5805 may be provided as the boot 5801 around the hard plastic part 5803. The soft part 5804 may be transparent and made of silicone. As depicted in FIG. 24B, when the end effector 5800 is pressed against the parenchyma 5850, the soft silicone part 5804 is pushed backwards, and the electrodes 5802 are exposed. FIG. 24C is another illustration of the end effector 5800 with the ribs version boot 5801 and a mock tissue 5860.

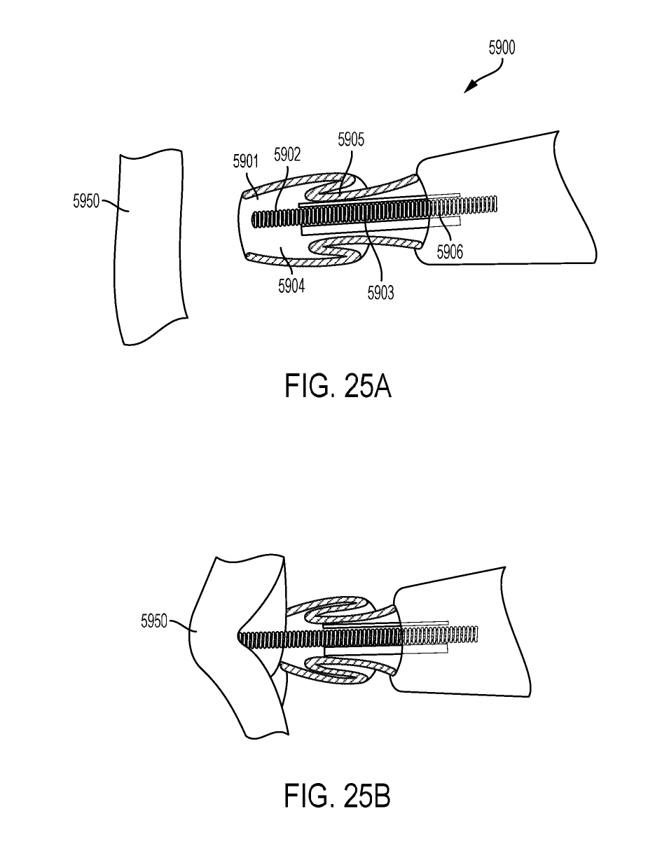

FIGS. 25A,B illustrate an S-wave version of the boot 5901. The end effector 5900 may include a hard plastic part 5903 within which the electrodes 5902 and fluid lumen 5906 are provided. The electrodes 5902 extends outside of the hard plastic part 5903. A soft part 5904 with an S-wave portion 5905 may be provided as the boot 5901 around the hard plastic part 5903. The soft part 5904 may be transparent and made of silicone. As depicted in FIG. 59(B), when the end effector 5900 is pressed against the parenchyma 5950, the soft silicone part 5904 is pushed backwards, and the electrodes 5902 are exposed.

Disclosed above are examples of surgical devices all of which are configured to seal tissue by the application of energy, and in which the surgical devices further include mechanisms and/or components to deliver material to and evacuate material from a surgical site wherein the tissue is sealed. While examples disclosed above include surgical devices having jaws or exposed electrodes, it may be understood that other surgical devices may be included without limitation, including those that contain one or more jaws and clamps, and/or one or more exposed, shrouded, or partially shrouded electrodes. In some examples, such surgical devices may include a combination or combinations of any one or more of such jaws, clamps, and exposed, shrouded, or partially shrouded electrodes.

Mechanisms and/or components to deliver material to the surgical site may include, without limitation, any one or more, or combination of one or more, devices configured to regulate the delivery of the material and devices configured to conduct the material to the surgical site. Mechanisms and/or components to regulate the delivery of material to the surgical site may include, without limitation, any one or more, or combination of one or more, valves, switches, or other devices configured to regulate the delivery of the material. Such regulation devices may be manually operable or electromechanically operable. Mechanisms and/or components to conduct the material to the surgical site may include, without limitation, any one or more, or combination of one or more, tubes, lumens, cannulae, or other devices configured to conduct the material to the surgical site. Such mechanisms and/or components to regulate the delivery of material or to conduct the material to the surgical site may be integral within a body of the surgical device, formed as part of a housing of the surgical device, attached to an exterior portion of the surgical device, separable from the surgical device, or any combination thereof. It may be recognized that all such mechanisms and/or components may be fabricated from any one or more materials appropriate for the function of the mechanisms and/or components.

Mechanisms and/or components to remove material from the surgical site may include, without limitation, any one or more, or combination of one or more, devices configured to regulate the evacuation of the material from the surgical site and devices configured to conduct the material from the surgical site. Mechanisms and/or components to regulate the evacuation of material from the surgical site may include, without limitation, any one or more, or combination of one or more, valves, switches, or other devices configured to regulate the evacuation of the material. Such regulation devices may be manually operable or electromechanically operable. Mechanisms and/or components to conduct material from the surgical site may include, without limitation, any one or more, or combination of one or more, tubes, lumens, cannulae, or other devices configured to conduct the material from the surgical site. Such mechanisms and/or components to regulate the evacuation of material or to conduct the material from the surgical site may be integral within a body of the surgical device, formed as part of a housing of the surgical device, attached to an exterior portion of the surgical device, separable from the surgical device, or any combination thereof. It may be recognized that such mechanisms and/or components may be fabricated from any one or more materials appropriate for the function of the mechanisms and/or components.

It may be recognized that any one or more mechanisms and/or components may be used to both deliver material to and evacuate material from the surgical site. For example, a three-way valve may be used to connect a common conducting device (such as a lumen) to either a source of a material to the surgical site or a sink of material from the surgical site.

The any one or more mechanisms and/or components used to regulate both the delivery of material to and evacuation of material from the surgical site may be controlled by one or more control devices. Such control devices may include mechanical control devices, for example, without limitation, switches, push buttons, slide actuators, and rotatory actuators. Any one or more of the mechanical control devices may operate to control one or more of the delivery of material to and evacuation of material from the surgical site. Such control devices may also include automated control devices that may operate under the direction of instructions provided to a computerized control device.

Material delivered to a surgical site may include any material having desirable properties for delivery to the surgical site. Desirable properties may include, without limitation, cooling the tissue, washing an energized component of the surgical device, or providing a material to help remove debris from the surgical site. Such materials may include, without limitation, fluids, solutions, suspensions, gases, and combination or combinations thereof. Examples of such materials may include, without limitation, distilled water, buffered saline, antibiotic solutions or suspensions, or water vapor. Material evacuated from a surgical site may include any material that may be unwanted at the surgical site. Unwanted material may include any material that may prevent, inhibit, or interfere with the surgical procedure. Such materials may include, without limitation, fluids, solutions, suspensions, particulates, and gases. Examples of such materials may include, without limitation, aqueous solutions, blood, suspensions of loose tissue, or water vapor.

While various details have been set forth in the foregoing description, it will be appreciated that the various aspects of the techniques for operating a generator for digitally generating electrical signal waveforms and surgical instruments may be practiced without these specific details. One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

Further, while several forms have been illustrated and described, it is not the intention of the applicant to restrict or limit the scope of the appended claims to such detail. Numerous modifications, variations, changes, substitutions, combinations, and equivalents to those forms may be implemented and will occur to those skilled in the art without departing from the scope of the present disclosure. Moreover, the structure of each element associated with the described forms can be alternatively described as a means for providing the function performed by the element. Also, where materials are disclosed for certain components, other materials may be used. It is therefore to be understood that the foregoing description and the appended claims are intended to cover all such modifications, combinations, and variations as falling within the scope of the disclosed forms. The appended claims are intended to cover all such modifications, variations, changes, substitutions, modifications, and equivalents.

For conciseness and clarity of disclosure, selected aspects of the foregoing disclosure have been depicted in block diagram form rather than in detail. Some portions of the detailed descriptions provided herein may be presented in terms of instructions that operate on data that is stored in one or more computer memories or one or more data storage devices (e.g. floppy disk, hard disk drive, Compact Disc (CD), Digital Video Disk (DVD), or digital tape). Such descriptions and representations are used by those skilled in the art to describe and convey the substance of their work to others skilled in the art. In general, an algorithm refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

Unless specifically stated otherwise as apparent from the foregoing disclosure, it is appreciated that, throughout the foregoing disclosure, discussions using terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. In one form, several portions of the subject matter described herein may be implemented via an application specific integrated circuits (ASIC), a field programmable gate array (FPGA), a digital signal processor (DSP), or other integrated formats. However, those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

In some instances, one or more elements may be described using the expression “coupled” and “connected” along with their derivatives. It should be understood that these terms are not intended as synonyms for each other. For example, some aspects may be described using the term “connected” to indicate that two or more elements are in direct physical or electrical contact with each other. In another example, some aspects may be described using the term “coupled” to indicate that two or more elements are in direct physical or electrical contact. The term “coupled,” however, also may mean that two or more elements are not in direct contact with each other, but yet still co-operate or interact with each other. It is to be understood that depicted architectures of different components contained within, or connected with, different other components are merely examples, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated also can be viewed as being “operably connected,” or “operably coupled,” to each other to achieve the desired functionality, and any two components capable of being so associated also can be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components, and/or electrically interacting components, and/or electrically interactable components, and/or optically interacting components, and/or optically interactable components.

In other instances, one or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

While particular aspects of the present disclosure have been depicted and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “one form,” or “a form” means that a particular feature, structure, or characteristic described in connection with the aspect is included in at least one aspect. Thus, appearances of the phrases “in one aspect,” “in an aspect,” “in one form,” or “in an form” in various places throughout the specification are not necessarily all referring to the same aspect. Furthermore, the particular features, structures or characteristics may be combined in any suitable manner in one or more aspects.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory).

A sale of a system or method may likewise occur in a territory even if components of the system or method are located and/or used outside the territory. Further, implementation of at least part of a system for performing a method in one territory does not preclude use of the system in another territory.

All of the above-mentioned U.S. patents, U.S. patent application publications, U.S. patent applications, foreign patents, foreign patent applications, non-patent publications referred to in this specification and/or listed in any Application Data Sheet, or any other disclosure material are incorporated herein by reference, to the extent not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

Various aspects of the subject matter described herein are set out in the following numbered examples:

### Example 1

A surgical instrument comprising:
a handle assembly;
a shaft coupled to a distal end of the handle assembly; and
an end effector coupled to a distal end of the shaft, the end effector comprising:
   a first jaw;
   a second jaw, wherein the first jaw and the second jaw cooperate to capture tissue therebetween;
   wherein at least one of the first and second jaws is configured to transmit electrosurgical energy to coagulate the tissue;
   an evacuation mechanism configured to evacuation fluid; and
   an irrigation mechanism configured to transmit fluid.

### Example 2

The surgical instrument of Example 1, wherein the first jaw comprises the evacuation mechanism and the irrigation mechanism, and the second jaw comprises a surface configured to transmit the electrosurgical energy upon contact with the tissue.

### Example 3

The surgical instrument of Example 2, wherein the first jaw comprises a tube running a longitudinal length of the first jaw, a distal end of the tube defining an evacuation and irrigation outlet on a distal end of the first jaw whereby fluid passes in or out of the first jaw.

### Example 4

The surgical instrument of Example 3, wherein the tube comprises at least one irrigation and evacuation outlet positioned on a lateral side of the first jaw.

### Example 5

The surgical instrument of any one or more of Examples 1 through 4, wherein the first jaw is configured to transmit electrosurgical energy at a first polarity and the second jaw is configured to transmit electrosurgical energy at a second polarity.

### Example 6

The surgical instrument of Example 5, wherein the first jaw or the second jaw comprises at least one insulating pin protruding on an inner side of said first or second jaw facing the other second or first jaw such that the at least one insulating pin is configured to touch the other second or first jaw upon closure of the first and second jaws and prevent direct contact between the first and second jaws, the at least one insulating pin configured to prevent energy transfer between the first and second jaws.

### Example 7

The surgical instrument of any one or more of Examples 1 through 6, wherein the end effector further comprises an insulated member positioned between the first and second jaws and is configured to isolate energy transfer between the first and second jaws.

### Example 8

The surgical instrument of Example 7, wherein the insulated member comprises an evacuation and irrigation channel configured to evacuation fluid entering the end effector and transmit fluid into the end effector.

### Example 9

The surgical instrument of Example 8, wherein the first and second jaws define an elongated fluid channel therebetween upon closure of the first and second jaws, wherein:
a distal end of the elongated channel defines an evacuation and irrigation outlet on a distal end of the first and second jaws whereby fluid passes in or out of the first and second jaws, and
a proximal end of the elongated channel is fluidically coupled to the evacuation and irrigation channel of the insulated member.

### Example 10

The surgical instrument of Example 9, wherein the evacuation mechanism and the irrigation mechanism are defined in part by the elongated fluid channel upon closure of the first and second jaws.

### Example 11

The surgical instrument of any one or more of Examples 8 through 10, wherein the insulated member is configured to be translatable along a longitudinal axis of the shaft.

### Example 12

The surgical instrument of any one or more of Examples 9 through 10, wherein translation of the insulated member in a distal direction along the longitudinal axis is configured to cause the first and second jaws to open, and translation of the insulated member in a proximal direction along the longitudinal axis is configured to cause the first and second jaw to close.

### Example 13

The surgical instrument of any one or more of Examples 1 through 12, wherein the electrosurgical energy is monopolar.

### Example 14

The surgical instrument of any one or more of Examples 1, through 12 wherein the electrosurgical energy is bipolar.

### Example 15

The surgical instrument of any one or more of Examples 1 through 14, wherein the first jaw comprises a backside positioned on a far side from the second jaw, the backside comprising an electrosurgical pad configured to transmit electrosurgical energy for coagulating tissue upon contact with the tissue.

### Example 16

A surgical instrument comprising:
a handle assembly;
a shaft coupled to a distal end of the handle assembly; and
an end effector coupled to a distal end of the shaft, the end effector comprising:
   an ultrasonic grasping member configured to contact tissue at a surgical site and transmit ultrasonic energy to the tissue upon contact;
   an evacuation mechanism configured to evacuation fluid; and
   an irrigation mechanism configured to transmit fluid.

### Example 17

The surgical instrument of Example 16, wherein the ultrasonic grasping member comprises a spoon-shaped distal end.

### Example 18

The surgical instrument of any one or more of Examples 16 through 17, wherein the end effector further comprises an evacuation and irrigation tube, the evacuation and irrigation tube partially defining the evacuation mechanism and the irrigation mechanism.

### Example 19

The surgical instrument of any one or more of Examples 16 through 18, wherein at least one of the ultrasonic grasping member, the evacuation mechanism and the irrigation mechanism is configured to retract into the shaft.

### Example 20

A surgical instrument comprising:
a handle assembly;
a shaft coupled to a distal end of the handle assembly; and
an end effector coupled to a distal end of the shaft, the end effector comprising:
   an outer tube coupled to the shaft;
   an inner tube positioned within the outer tube and coupled to an inner tube of the shaft, the inner tube comprising a grasping and sealing mechanism configured to grasp tissue and transmit energy to coagulate the tissue upon contact;
   an evacuation and irrigation mechanism defined in part by a space in between the inner tube and the outer tube, wherein the evacuation and irrigation mechanism is configured to:
      evacuation fluid from a distal end of the end effector through the space in between the inner tube and the outer tube, and
      transmit fluid through the distal end of the effector from the space in between the inner tube and the outer tube.

### Example 21

The surgical instrument of any one or more of Examples 1 through 15, further comprising a clamp arm coupled to the first jaw and configured to open the first jaw about a hinge coupled to the shaft.

### Example 22

The surgical instrument of Example 21, further comprising a spring torsion system configured to close the first jaw onto the second jaw while no force is exerted on the clamp arm.

### Example 23

The surgical instrument of Example 22, further comprising a rotatable mechanical bar pivotally coupled to the clamp arm on a first end and pivotally coupled to a horizontal bar of the spring torsion system on the second end opposite the first end.

### Example 24

The surgical instrument of Example 23, wherein the rotatable mechanical bar is positioned at a first angle greater than 45 degrees and less than 90 degrees from horizontal when the first jaw is closed onto the second jaw.

### Example 25

The surgical instrument of Example 23, wherein the rotatable mechanical bar is positioned at a second angle less than 45 degrees and greater than 0 degrees from horizontal when the clamped arm is pressed and the first jaw is in an open position.

### Example 26

A control system of a surgical instrument, comprising:
a fluid source;
a power generator;
a pump coupled to the power generator and fluidically coupled to the fluid source;
a valve;
a vacuum source coupled to the valve;
a controller coupled to the valve; and
the surgical instrument coupled to the fluid source via a first fluid line, the pump via a second fluid line, the vacuum source via a first vacuum line, the valve via a second vacuum line, the generator via a first electrical line, and the controller via a second electrical line.

### Example 27

The control system of Example 26, wherein the surgical instrument comprises a first button configured to control the generator to cause the pump to deliver a modulated drip functionality of fluid to the surgical instrument from the fluid source via the second fluid line, and simultaneously cause the controller to control the valve to deliver a pulsing vacuum function to the surgical instrument from the vacuum source via the second vacuum line.

### Example 28

The control system of Example 27, further comprising a second button configured to cause an uninterrupted vacuum functionality directly to the surgical instrument from the vacuum source via the first vacuum line.

### Example 29

The control system of Example 27, further comprising a third button configured to cause an uninterrupted fluid flow functionality directly to the surgical instrument from the fluid source via the first fluid line.

### Example 30

The control system of Example 27, wherein a rate of drip of the fluid in the modulated drip functionality is controlled by the generator and is directly proportional to a rate of radio frequency (RF) energy delivered by the generator to the surgical instrument.

### Example 31

The control system of Example 27, wherein a rate of vacuum pulsing of the pulsing vacuum function is controlled by the controller and is directly proportional to a rate of radio frequency (RF) energy delivered by the generator to the surgical instrument.

### Example 32

A surgical device, comprising:
a handle comprising a power control member, an irrigation control member, and an evacuation control member;
an end effector comprising two electrodes of opposite polarities, an evacuation lumen, and an irrigation lumen; and
an elongate member connecting the handle and the end effector.

### Example 33

The surgical device of Example 32, wherein the irrigation lumen comprises slots on side surfaces of the two electrodes.

### Example 34

The surgical device of any one or more of Examples 32 through 33, wherein the irrigation lumen comprises slots provided on a probe between the two electrodes.

### Example 35

The surgical device of any one or more of Examples 32 through 34, wherein the irrigation lumen comprises a plurality of irrigation outlets comprising at least one lateral irrigation outlet and at least one distal irrigation outlet.

### Example 36

A surgical device, comprising:
a handle comprising a power control member, an irrigation control member, and an evacuation control member;
an end effector comprising at least two electrodes of opposite polarities, an evacuation lumen, and an irrigation lumen;
a boot around the end effector; and
an elongate member connecting the handle and the end effector.

### Example 37

The surgical device of Example 36, wherein the boot comprises an opening on a side surface of the boot.

### Example 38

The surgical device of any one or more of Examples 36 through 37, wherein the boot comprises one or more ribs on a side surface.

### Example 39

The surgical device of any one or more of Examples 36 through 38, wherein the boot comprises an S-wave portion on a side surface.

### Example 40

A surgical device, comprising:
a handle comprising a power control member, an irrigation control member, and an evacuation control member;
an end effector comprising at least two electrodes of opposite polarities, an evacuation lumen, and an irrigation lumen; and
an elongate member connecting the handle and the end effector,
wherein the end effector is connected to a spring in an interior of the elongate member, the spring causing the end effector to move along a longitudinal axis of the elongate member.

### Example 41

The surgical device of Example 40, wherein the evacuation lumen comprises a slot, the spring is connected to a pin, and the pin slidably fits in the slot.

### Example 42

The surgical device of any one or more of Examples 40 through 41, wherein the evacuation lumen extends about 1-2 mm longer than the two electrodes when the spring is in a relaxed state.

### Example 43

The surgical device of any one or more of Examples 40 through 42, wherein the power control member is activated electrically or mechanically.

### Example 44

An end effector, comprising:
three electrodes;
an evacuation lumen; and
an irrigation lumen.

### Example 45

The end effector of Example 44, wherein the three electrodes comprise a positive electrode, a negative electrode.

### Example 46

The end effector of any one or more of Examples 44 through 45, wherein the irrigation lumen comprises a plurality of irrigation outlets on a side of the end effector.

### Example 47

The end effector of any one or more of Examples 44 through 46, wherein the evacuation lumen is provided on a distal face of the end effector.

### Example 48

A surgical device, comprising:
an end effector comprising two electrodes separable from each other, an evacuation lumen, and an irrigation lumen;
a handle comprising a power control member, an irrigation control member, an evacuation control member, an electrode spreading control member;
an elongate member connecting the handle and the end effector; and
a control mechanism configured to control spreading of the two electrodes.

### Example 49

The surgical device of Example 48, wherein the control mechanism comprises a shaft configured to connect the electrode spreading control member and the two electrodes.

### Example 50

The surgical device of Example 49, wherein the two electrodes are pivotably connected to each other, one of the two electrodes comprises a slot, the shaft comprises a pin, and the pin fits in the slot and slides along the slot when the shafts moves along a longitudinal axis of the elongate member.

### Example 51

The surgical device of any one or more of Examples 48 through 50, wherein the control mechanism comprises a shaft and a cam block, the cam block comprising two slots, the two electrodes each comprise a pin, each pin fitting in each slot and sliding along each slot when the shaft moves along the elongate member.

### Example 52

An end effector, comprising:
two helical electrodes of opposite polarities;
an evacuation lumen; and
an irrigation lumen.

### Example 53

The end effector of Example 52, wherein the evacuation lumen is provided at a tip of the end effector.

### Example 54

The end effector of any one or more of Examples 52 through 53, wherein the irrigation lumen comprises fluid outlets provided around the evacuation lumen.

### Example 55

The end effector of any one or more of Examples 52 through 54, wherein the irrigation lumen comprises fluid outlets provided at a proximal end of the end effector.

### Example 56

An end effector, comprising:
two electrodes of opposite polarities;
an evacuation lumen; and
one or more irrigation channels.

### Example 57

The end effector of Example 56, wherein the evacuation lumen is provided at a tip of the end effector.

### Example 58

The end effector of any one or more of Examples 56 through 57, further comprising flow directing members.

### Example 59

The end effector of any one or more of Examples 56 through 58, further comprising at least one side evacuation slot.

### Example 60

A surgical device assembly, comprising:
an end effector and shaft assembly comprising an end effector, a shaft, and a first interface, the end effector comprising two electrodes, an evacuation lumen, and an irrigation lumen; and
a handle assembly comprising a handle and a lumen cartridge, the handle comprising a power control member, an irrigation control member, an evacuation control member, and a second interface, and the lumen cartridge configured to fit in a bottom portion of the handle.

### Example 61

The surgical device assembly of Example 60, wherein the first and second interfaces each comprise a connecting member, a conducting member, and two holes for irrigation and evacuation tubes.

### Example 62

The surgical device assembly of Example 61, wherein the connecting member comprises one or more magnets.

### Example 63

The surgical device assembly of Example 61, wherein the connecting member is conductive and electrically connected to the conducting member.

### Example 64

A surgical instrument comprising:
a handle assembly;
a shaft coupled to a distal end of the handle assembly;
an end effector coupled to a distal end of the shaft, the end effector comprising:
a first jaw;
a second jaw, wherein the first jaw and the second jaw cooperate to capture tissue therebetween and wherein at least one of the first and second jaws is configured to transmit electrosurgical energy to coagulate the tissue;
an evacuation mechanism configured to evacuate fluid; and
an irrigation mechanism configured to transmit fluid; and
a clamp arm coupled to the first jaw and configured to open the first jaw about a hinge coupled to the shaft.

### Example 65

The surgical instrument of Example 64, further comprising a spring torsion system configured to close the first jaw onto the second jaw while no force is exerted on the clamp arm.

### Example 66

The surgical instrument of Example 65, further comprising a rotatable mechanical bar pivotally coupled to the clamp arm on a first end and pivotally coupled to a horizontal bar of the spring torsion system on the second end opposite the first end.

### Example 67

The surgical instrument of Example 66, wherein the rotatable mechanical bar is positioned at a first angle greater than 45 degrees and less than 90 degrees from horizontal when the first jaw is closed onto the second jaw.

### Example 68

The surgical instrument of Example 66, wherein the rotatable mechanical bar is positioned at a second angle less than 45 degrees and greater than 0 degrees from horizontal when the clamped arm is pressed and the first jaw is in an open position.

### Example 69

A control system of a surgical instrument, comprising:
a fluid source;
a power generator;
a pump coupled to the power generator and fluidically coupled to the fluid source;
a valve;
a vacuum source coupled to the valve; and
a controller coupled to the valve,
wherein the surgical instrument is fluidically coupled to the fluid source via a first fluid line,
wherein the surgical instrument is fluidically coupled to the pump via a second fluid line,
wherein the surgical instrument is fluidically coupled to the vacuum source via a first vacuum line,
wherein the surgical instrument is fluidically coupled to the valve via a second vacuum line,
wherein the surgical instrument is electrically coupled to the generator via a first electrical line, and
wherein the surgical instrument is electrically coupled to the controller via a second electrical line.

### Example 70

The control system of Example 69, wherein the surgical instrument comprises a first button configured to control the generator to cause the pump to deliver a modulated drip functionality of fluid to the surgical instrument from the fluid source via the second fluid line, and simultaneously cause the controller to control the valve to deliver a pulsing vacuum function to the surgical instrument from the vacuum source via the second vacuum line.

### Example 71

The control system of Example 70, further comprising a second button configured to cause an uninterrupted vacuum functionality directly to the surgical instrument from the vacuum source via the first vacuum line.

### Example 72

The control system of any one or more of Example 70 through Example 71, further comprising a third button configured to cause an uninterrupted fluid flow functionality directly to the surgical instrument from the fluid source via the first fluid line.

### Example 73

The control system of any one or more of Example 70 through Example 72, wherein a rate of drip of the fluid in the modulated drip functionality is controlled by the generator and is directly proportional to a rate of radio frequency (RF) energy delivered by the generator to the surgical instrument.

### Example 74

The control system of any one or more of Example 70 through Example 73, wherein a rate of vacuum pulsing of the pulsing vacuum function is controlled by the controller and is directly proportional to a rate of radio frequency (RF) energy delivered by the generator to the surgical instrument.

### Example 75

A surgical device, comprising:
a handle comprising a power control member, an irrigation control member, and an evacuation control member;
an end effector comprising two electrodes of opposite polarities, an evacuation lumen, and an irrigation lumen; and
an elongate member connecting the handle and the end effector.

### Example 76

The surgical device of Example 75, having an effective distance between a distal end of one or both of the two electrodes and a distal end of the evacuation lumen.

### Example 77

The surgical device of Example 76, wherein the distal end of the evacuation lumen is located proximally to the distal end of the one or both of the two electrodes, and wherein the effective distance has a range of 0.1 inch to 0.5 inches.

### Example 78

The surgical device of Example 76, wherein the distal end of the evacuation lumen is located proximally to the distal end of the one or both of the two electrodes, and wherein the effective distance has a range of 0.2 inches to 0.4 inches.

### Example 79

The surgical device of Example 76, wherein the distal end of the evacuation lumen is located proximally to the distal end of the one or both of the two electrodes, and wherein the effective distance has a range of 0.3 inches.

### Example 80

A surgical device, comprising:
a handle comprising a power control member, an irrigation control member, and an evacuation control member;
an end effector comprising at least two electrodes of opposite polarities, an evacuation lumen, and an irrigation lumen;
a boot around the end effector; and
an elongate member connecting the handle and the end effector.

### Example 81

The surgical device of Example 80, wherein the boot comprises an opening on a side surface of the boot.

### Example 82

The surgical device of any one or more of Example 80 through Example 81, wherein the boot comprises one or more ribs on a side surface.

### Example 83

The surgical device of Example 80 through Example 82, wherein the boot comprises an S-wave portion on a side surface.

### Example 84

The surgical device of Example 80 through Example 83, wherein the boot comprises a shape that is configured to change to conform to a tissue surface when pressed thereagainst.

### Example 85

An end effector of a surgical device comprising:
a first jaw;
a second jaw,
wherein the first jaw and the second jaw cooperate to capture a tissue therebetween and wherein at least one of the first jaw and the second jaw is configured to transmit electrosurgical energy to coagulate the tissue;
a closure saddle in mechanical communication with the first jaw;
a closure tube in mechanical communication with the closure saddle,
wherein the closure saddle is configured to adjust a position of the first jaw with respect to the second jaw based on a position of the closure tube;
an evacuation mechanism configured to evacuate fluid; and
an irrigation mechanism configured to transmit fluid.

### Example 86

The end effector of Example 85, wherein the position of the first jaw with respect to the second jaw is a closed position when the closure tube is in a proximal position.

### Example 87

The end effector of any one or more of Example 85 through Example 86, wherein the position of the first jaw with respect to the second jaw is an open position when the closure tube is in a distal position.

### Example 88

The end effector of any one or more of Example 85 through Example 87, wherein the closure tube is configured to translate in a longitudinal direction.

### Example 89

The end effector of any one or more of Example 85 through Example 88, further comprising a pivot pin in mechanical communication with the first jaw wherein the first jaw is configured to pivot about an axis of the pivot pin.

### Example 90

The end effector of any one or more of Example 85 through Example 89, further comprising a cam configured to engage a portion of the closure saddle.

### Example 91

The end effector of any one or more of Example 85 through Example 90, wherein at least one of the first jaw and the second jaw comprises one or more insulated pins configured to separate and insulate the first jaw and the second jaw when the first jaw and the second jaw are in a closed position.

What is claimed is:

1. A surgical instrument comprising:
a handle assembly;
a shaft coupled to a distal end of the handle assembly;
an end effector coupled to a distal end of the shaft, the end effector comprising:
a first jaw;
a second jaw, wherein the first jaw and the second jaw cooperate to capture tissue therebetween and wherein at least one of the first and second jaws is configured to transmit electrosurgical energy to coagulate the tissue;
an evacuation mechanism configured to evacuate fluid; and
an irrigation mechanism configured to transmit fluid; and
a clamp arm coupled to the first jaw and configured to open the first jaw about a hinge coupled to the shaft.

2. The surgical instrument of claim 1, further comprising a spring torsion system configured to close the first jaw onto the second jaw while no force is exerted on the clamp arm.

3. The surgical instrument of claim 2, further comprising a rotatable mechanical bar pivotally coupled to the clamp arm on a first end and pivotally coupled to a horizontal bar of the spring torsion system on the second end opposite the first end.

4. The surgical instrument of claim 3, wherein the rotatable mechanical bar is positioned at a first angle greater than 45 degrees and less than 90 degrees from horizontal when the first jaw is closed onto the second jaw.

5. The surgical instrument of claim 3, wherein the rotatable mechanical bar is positioned at a second angle less than 45 degrees and greater than 0 degrees from horizontal when the clamped arm is pressed and the first jaw is in an open position.

6. A control system of a surgical instrument, comprising:
a fluid source;
a power generator;
a pump coupled to the power generator and fluidically coupled to the fluid source;
a valve;
a vacuum source coupled to the valve; and
a controller coupled to the valve,
wherein the surgical instrument is fluidically coupled to the fluid source via a first fluid line,
wherein the surgical instrument is fluidically coupled to the pump via a second fluid line,
wherein the surgical instrument is fluidically coupled to the vacuum source via a first vacuum line,
wherein the surgical instrument is fluidically coupled to the valve via a second vacuum line,
wherein the surgical instrument is electrically coupled to the generator via a first electrical line, and
wherein the surgical instrument is electrically coupled to the controller via a second electrical line.

7. The control system of claim 6, wherein the surgical instrument comprises a first button configured to control the generator to cause the pump to deliver a modulated drip functionality of fluid to the surgical instrument from the fluid source via the second fluid line, and simultaneously cause the controller to control the valve to deliver a pulsing vacuum function to the surgical instrument from the vacuum source via the second vacuum line.

8. The control system of claim 7, further comprising a second button configured to cause an uninterrupted vacuum functionality directly to the surgical instrument from the vacuum source via the first vacuum line.

9. The control system of claim 7, further comprising a third button configured to cause an uninterrupted fluid flow functionality directly to the surgical instrument from the fluid source via the first fluid line.

10. The control system of claim 7, wherein a rate of drip of the fluid in the modulated drip functionality is controlled by the generator and is directly proportional to a rate of radio frequency (RF) energy delivered by the generator to the surgical instrument.

11. The control system of claim 7, wherein a rate of vacuum pulsing of the pulsing vacuum function is controlled by the controller and is directly proportional to a rate of radio frequency (RF) energy delivered by the generator to the surgical instrument.

12. A surgical device, comprising:
a handle comprising a power control member, an irrigation control member, and an evacuation control member;
an end effector comprising two electrodes of opposite polarities, an evacuation lumen, and an irrigation lumen; and
an elongate member connecting the handle and the end effector.

13. The surgical device of claim 12, having an effective distance between a distal end of one or both of the two electrodes and a distal end of the evacuation lumen.

14. The surgical device of claim 13, wherein the distal end of the evacuation lumen is located proximally to the distal end of the one or both of the two electrodes, and wherein the effective distance has a range of 0.1 inch to 0.5 inches.

15. The surgical device of claim 13, wherein the distal end of the evacuation lumen is located proximally to the distal end of the one or both of the two electrodes, and wherein the effective distance has a range of 0.2 inches to 0.4 inches.

16. The surgical device of claim 13, wherein the distal end of the evacuation lumen is located proximally to the distal end of the one or both of the two electrodes, and wherein the effective distance has a range of 0.3 inches.

17. A surgical device, comprising:
a handle comprising a power control member, an irrigation control member, and an evacuation control member;
an end effector comprising at least two electrodes of opposite polarities, an evacuation lumen, and an irrigation lumen;
a boot around the end effector; and
an elongate member connecting the handle and the end effector.

18. The surgical device of claim 17, wherein the boot comprises an opening on a side surface of the boot.

19. The surgical device of claim 17, wherein the boot comprises one or more ribs on a side surface.

20. The surgical device of claim 17, wherein the boot comprises an S-wave portion on a side surface.

21. The surgical device of claim 17, wherein the boot comprises a shape that is configured to change to conform to a tissue surface when pressed thereagainst.

22. An end effector of a surgical device comprising:
a first jaw;
a second jaw,
wherein the first jaw and the second jaw cooperate to capture a tissue therebetween and wherein at least one of the first jaw and the second jaw is configured to transmit electrosurgical energy to coagulate the tissue;
a closure saddle in mechanical communication with the first jaw;
a closure tube in mechanical communication with the closure saddle,
wherein the closure saddle is configured to adjust a position of the first jaw with respect to the second jaw based on a position of the closure tube;
an evacuation mechanism configured to evacuate fluid; and
an irrigation mechanism configured to transmit fluid.

23. The end effector of claim 22, wherein the position of the first jaw with respect to the second jaw is a closed position when the closure tube is in a proximal position.

24. The end effector of claim 22, wherein the position of the first jaw with respect to the second jaw is an open position when the closure tube is in a distal position.

25. The end effector of claim 22, wherein the closure tube is configured to translate in a longitudinal direction.

26. The end effector of claim 22, further comprising a pivot pin in mechanical communication with the first jaw wherein the first jaw is configured to pivot about an axis of the pivot pin.

27. The end effector of claim 22, further comprising a cam configured to engage a portion of the closure saddle.

28. The end effector of claim 22, wherein at least one of the first jaw and the second jaw comprises one or more insulated pins configured to separate and insulate the first jaw and the second jaw when the first jaw and the second jaw are in a closed position.
